(19) United States
(12) Patent Application Publication
(10) Pub. No.: US 2018/0155419 A1
(43) Pub. Date: Jun. 7, 2018

(54) ANTI-MIF ANTIBODIES IN THE TREATMENT OF CANCERS CONTAINING MUTANT TP53 AND/OR MUTANT RAS

(71) Applicants: Baxalta GmbH, Zug (CH); Baxalta Incorporated, Bannockburn, IL (US)

(72) Inventors: Patrice Douillard, Wien (AT); Randolf Kerschbaumer, Klosterneuburg (AT); Michael Thiele, Wien (AT); Salim Yazji, Highland Park, IL (US)

(21) Appl. No.: 15/575,356

(22) PCT Filed: May 18, 2016
(86) PCT No.: PCT/EP2016/061087

§ 371 Date: Nov. 18, 2017

Related U.S. Application Data
(60) Provisional application No. 62/163,263, filed on May 18, 2015.

(51) Int. Cl. C07K 16/24; A61K 39/395; A61P 35/00

(52) U.S. Cl. CPC C07K 16/24; A61K 2039/505; A61P 35/00; A61K 39/39566

(57) ABSTRACT

The present invention pertains to anti-MIF antibodies, preferably in combination with cancer therapeutics, i.e. chemotherapeutic agents, in the treatment of cancers containing mutant TP53 and/or mutant RAS.

### BACKGROUND

Macrophage migration inhibitory factor (MIF) is a cytokine initially isolated based upon its ability to inhibit the in vitro random migration of peritoneal exudate cells from tuberculin hypersensitive guinea pigs (containing macrophages) (Bloom et al. Science 1966, 153, 80-2; David et al. PNAS 1966, 56, 72-7). Today, MIF is known as a critical upstream regulator of the innate and acquired immune response that exerts a pleiotropic spectrum of activities.

The human MIF cDNA was cloned in 1989 (Weiser et al., PNAS 1989, 86, 7522-6), and its genomic localization was mapped to chromosome 22. The product of the human MIF gene is a protein with 114 amino acids (after cleavage of the N-terminal methionine) and an apparent molecular mass of about 12.5 kDa. MIF has no significant sequence homology to any other protein. The protein crystallizes as a trimer of identical subunits. Each monomer contains two antiparallel alpha-helices that pack against a four-stranded beta-sheet. The monomer has additional two beta-strands that interact with the beta-sheets of adjacent subunits to form the interface between monomers. The three subunits are arranged to form a barrel containing a solvent-accessible channel that runs through the center of the protein along a molecular three-fold axis (Sun et al. PNAS 1996, 93, 5191-5196).

It was reported that MIF secretion from macrophages was induced at very low concentrations of glucocorticoids (Calandra et al. Nature 1995, 377, 68-71). However, MIF also counter-regulates the effects of glucocorticoids and stimulates the secretion of other cytokines such as tumor necrosis factor TNF-α and interleukin IL-1 β (Baugh et al., Crit Care Med 2002, 30, S27-35). MIF was also shown e.g. to exhibit pro-angiogenic, pro-proliferative and anti-apoptotic properties, thereby promoting tumor cell growth (Mitchell, R.A., Cellular Signalling, 2004. 16(1): p. 13-19; Lue, H. et al., Oncogene 2007. 26(35): p. 5046-59). It is also e.g. directly associated with the growth of lymphoma, melanoma, and colon cancer (Nishihira et al. J Interferon Cytokine Res. 2000, 20:751-62).

MIF is a mediator of many pathologic conditions and thus associated with a variety of diseases including inter alia inflammatory bowel disease (IBD), rheumatoid arthritis (RA), acute respiratory distress syndrome (ARDS), asthma, glomerulonephritis, IgA nephropathy, myocardial infarction (MI), sepsis and cancer, though not limited thereto.

Polyclonal and monoclonal anti-MIF antibodies have been developed against recombinant human MIF (Shimizu et al., FEBS Lett. 1996; 381, 199-202; Kawaguchi et al, Leukoc. Biol. 1986, 39, 223-232, and Weiser et al., Cell. Immunol. 1985, 90, 167-78).

Anti-MIF antibodies have been suggested for therapeutic use. Calandra et al., (J. Inflamm. (1995); 47, 39-51) reportedly used anti-MIF antibodies to protect animals from experimentally induced gram-negative and gram-positive septic shock. Anti-MIF antibodies were suggested as a means of therapy to modulate cytokine production in septic shock and other inflammatory disease states.

U.S. Pat. No. 6,645,493 discloses monoclonal anti-MIF antibodies derived from hybridoma cells, which neutralize the biological activity of MIF. It could be shown in an animal model that these mouse-derived anti-MIF antibodies had a beneficial effect in the treatment of endotoxin-induced shock.

US 200310235584 discloses methods of preparing high affinity antibodies to MIF in animals in which the MIF gene has been homozygously knocked-out.

PCT/EP2013/057894 (published as WO2013/156473) discloses anti-MIF antibodies and their uses in combination with chemotherapeutic agents in the treatment of cancer.

Glycosylation-inhibiting factor (GIF) is a protein described by Galat et al. (Eur. J. Biochem, 1994, 224, 417-21). MIF and GIF are now recognized to be identical. Watarai et al. (PNAS 2000, 97, 13251-6) described polyclonal antibodies binding to different GIF epitopes to identify the biochemical nature of the posttranslational modification of GIF in Ts cells. Watarai et al, supra, reported that GIF occurs in different conformational isoforms in vitro. One type of isomer occurs by chemical modification of a single cysteine residue. The chemical modification leads to conformational changes within the GIF protein.

One of those medical diseases and disorders in which MIF has been implicated for the past decades is—as pointed out above—cancer. Cancer is generally treated via various routes, one of them being the use of so-called chemotherapeutic agents (which are the basis of anticancer chemotherapy). The concept underlying chemotherapy in the general sense thereof, posits that a disease or disorder (caused by bacteria, viruses, parasites and cancer cells) can be effectively treated by way of chemical compounds. One particular indication of chemotherapy is cancer. Chemotherapeutic agents can act for example by killing cells that divide more rapidly than other cells, and thus target cancer cells which commonly divide more rapidly than non-cancerous cells. Most chemotherapeutic agents drugs work by impairing cell division, i.e., they act at one or several stages of the cell cycle and thus are able to target cells that divide more rapidly. Chemotherapeutic agents can be either cytostatic, i.e. they slow down or abrogate the growth or division of cells; other chemotherapeutic drugs can cause damage to cells and kill them; in that case they are termed cytotoxic. Most cytotoxic drugs inflict a damage that per se does not suffice to kill a cell but that generates a stimulus to initiate programmed cell death (apoptosis).

In general, major classes of chemotherapeutic drugs are alkylating agents, anti-metabolites, anthracyclines, plant alkaloids, topoisomerases and other anti-tumour agents. Most commonly, as mentioned above, these drugs affect cell division; they can also affect DNA synthesis or function. Other chemotherapeutics do not directly interfere with DNA. These are newer classes of chemotherapeutic agents, which are referred to as signal interceptors, which include monoclonal antibodies and tyrosine kinase inhibitors like imatinib mesylate. Examples for alkylating agents, which alkylate nucleophilic functional groups are mechlorethamine, cyclophosphamide, chlorambucil, melphalane, trofosfamide, ifosfamide, carmustine, lomustine, dacarbazine, temozolomide, mitomycine C and many others. Cisplatin, carboplatin, oxaliplatin and other platinum containing compounds form stable complexes with DNA.

Cytotoxic anti-metabolites are folic acid analogues (e.g., methotrexate/aminopterin, raltitrexed, pemetrexed, or leucovorin (also termed folinic acid)), purines (e.g., 6-mercaptopurine, azathioprine, thioguanine, fludarabine, cladribine) or pyrimidines (cytarabine, gemcitabine, deazacytidine, 5-fluoruracil and its prodrugs including capecitabine). Antimetabolites either inhibit DNA-synthesis by interfering with crucial steps in the de novo synthesis of purine and pyrimidine nucleotides or they become incorporated into DNA during the S-phase of the cell cycle, where they interfere with DNA-folding, DNA-repair or methylation. Alternatively, some compounds also become incorporated into RNA.

Examples for alkaloids and terpenoids which are derived from plants and block cell division by preventing microtubule function are vinca-alkaloids and taxanes. Particularly well known vinca-alkaloids are vincristine, vinblastine, vinorelbine and vindesine. Podophyllotoxin is an additional example of a plant-derived compound. An example for a taxane is docetaxel or paclitaxel. Estramustin is an example of a synthetic compound that targets tubulin.

Examples of topoisomerase inhibitors, which are inhibitors of enzymes that maintain the topology of DNA, include camphtotecines like irinotecan and topotecan (type 1 topoisomerase inhibitors) or amsacrin, etoposide, etoposide phosphate and teniposide (topoisomerase-type 2 inhibitors).

Finally, examples of antineoplastic intercalating agents include dactinomycin, doxorubicin, epirubicin, bleomycin and others.

A comprehensive overview is comprised in Goodman and Gilman, The Pharmacological Basis of Therapeutics, 12th Edition, “General Principles of Cancer Chemotherapy. Introduction” as shown below. Several tumors are susceptible to hormone therapy: glucocorticoids (e.g., prednisolone, dexamethasone and may others) promote apoptosis of lymphoma cells. They are therefore included in typical chemotherapeutic regimen. Similarly, several types of cancer are susceptible to hormonal interventions. This includes, e.g., breast cancer, ovarian cancer and prostate cancer. Hormonal ablation can be achieved by suppressing pituitary release of gonadotropins with gonadotropin-releasing hormone receptor agonists (e.g., buserelin, goserelin, leuprolide, hisrelin etc.), which induce desensitization of the receptor and hence inhibit hormone production, or with gonadotropin-releasing hormone receptor antagonists (e.g., degarelix). Alternatively, the action of estrogens and of androgens may be blocked by hormone receptor antagonists: compounds that act as partial agonists at estrogen receptors (also referred to as selective estrogen receptor modulators, SERM's) include tamoxifen, raloxifen and toremifen. Fulvestrant is an example of a pure estrogen recepor antagonist. Androgen receptors can be blocked by antagonists such as flutamide, bicalitamide and cyproterone. Finally, hormonal ablation can be achieved by blocking the pertinent enzymes, which are responsible for their synthesis. In the case of estrogens, it is the aromatase (CYP19), which is blocked by compounds such as aminoglutethimide, formestane, exemestane, anastrazole and letrozole. Androgen production can be suppressed by inhibiting the enzyme 17 α-hydroxylase/C17,20 lyase (CYP17A1) with abiraterone. Regardless of by which approach hormonal input is blocked, the growth of susceptible cancer cells is suppressed and their apoptosis is promoted.

Chemotherapeutic agents have been shown to be useful and successful in the treatment of several different cancer types.

However, it has been found that sub-groups of cancers where particular cancer-related genes are mutated suffer from poor prognosis and can be more resistant to treatment than corresponding cancers which do not contain mutations in these genes.

For instance, the TP53 gene encodes a tumor suppressor protein, named p53, which exerts a variety of tumor suppressive effects as a wild-type protein (Vogelstein et al., Nature. 2000, 408(6810), 307-10), including a regulation of the cell cycle, an induction of apoptosis in response to DNA damage, an induction of DNA repair, an induction of senescence and an inhibition of angiogenesis. Due to its central role in exerting tumor suppressive effects, and in particular in exerting these effects in response to DNA damage, the p53 protein has also been referred to as the ‘guardian of the genome’. In humans, the TP53 gene is located on chromosome 17 (17p13.1). The p53 wild-type protein functions as a sequence-specific transcription factor that exerts its aforementioned effects by regulating transcription of a variety of target genes. Somatic mutations in the TP53 gene have been found in a variety of cancers. Typically, these TP53 mutations in human cancers are acquired by a somatic missense mutation in one allele, followed by a loss of heterozygosity (LOH) at the TP53 locus, which leads to a deletion of the wild-type TP53 allele and to a loss of the corresponding wild-type p53 protein. The mutant p53 protein that is expressed by the remaining allele cannot exert the tumor-suppressive functions of the wild-type protein. Additionally, a gain of cancer-promoting functions has been described for some p53 missense mutations (Freed-Pastor and Prives, Genes Dev. 2012, 26(12),1268-86). Notably, somatic mutations in the TP53 gene of the cancer cells have been found to be markers of poor prognosis in several cancers including breast cancer and colorectal cancer (Petitjean et al., Oncogene. 2007, 26(15), 2157-65; Kressner et al., J Clin Oncol. 1999,17(2), 593-9).

The KRAS gene is a proto-oncogene, which encodes the K-ras protein, a GTPase involved in signal transduction pathways. The K-ras protein is part of the Ras superfamily of proteins. The KRAS gene is located on human chromosome 12 and contains four coding exons and a 5′ exon which is non-coding. The wild-type K-ras protein can switch between an activated and an inactive state. K-ras protein can be activated by upstream signals, in particular by the binding of growth factors to their receptors such as binding of epidermal growth factor (EGF) to its receptors (EGFRs). In its activated state, the K-ras protein activates downstream signal transduction which leads to the phosphorylation of AKT and ERK, which in turn promote cell growth, cell proliferation and cell survival. When K-ras protein gets activated through either activating mutation or amplification of the KRAS gene in cancer, the downstream signaling pathways which promote cell growth, cell proliferation and cell survival will become constitutively activated and contribute to cancer pathogenesis. This renders the cancer resistant to drugs that target signals which are upstream of K-ras such as drugs to the epidermal growth factor receptors (EGFRs). Furthermore, it was more generally found that somatic mutations in the KRAS gene of the cancer cells are markers of poor prognosis in some cancers including colorectal cancer (CRC) and breast cancer (Pereira et al., PLoS One. 2013, 8(3), e60576; Phipps et al., British Journal of Cancer (2013) 108, 1757-1764).

Similarly, the NRAS gene is also a proto-oncogene, which encodes the N-ras protein, a GTPase involved in signal transduction pathways. Like the K-ras protein, the N-ras protein is also part of the Ras superfamily of proteins. The NRAS gene is located on human chromosome 1 and contains seven exons. Similar to the KRAS gene and the k-ras protein, the NRAS gene, the N-ras protein and their activation have also been implicated in cancers.

Therefore, there remains a need in the art for the provision of a therapy of cancer which allows to improve the treatment of cancers containing mutations which are associated with poor prognosis and to improve the treatment of cancers containing mutations which confer resistance to existing cancer therapies.

### DESCRIPTION OF THE INVENTION

This object has been solved by the present invention.

In particular it was surprisingly shown by the inventors that anti-MIF antibodies can be used to treat cancers where the tumor suppressor protein p53 has been inactivated by TP53 mutation. According to the invention, TP53 and/or RAS mutant cancers can be treated by anti-MIF antibodies as monotherapy, or they can be even more efficiently treated by a combination therapy of an anti-MIF antibody with a chemotherapeutic agent. The present invention provides advantageous uses of anti-MIF antibodies for the treatment of these RAS and/or TP53 mutant tumors, and for the specific treatment of effects caused by the mutant RAS gene and its K-ras or N-ras protein products.

Elevated MIF levels, i.e. levels of MIF in general are detected after the onset of various diseases, inter alia after the onset of cancer. However, MIF circulates also in healthy subjects, which makes a clear differentiation difficult. oxMIF, on the contrary, is not present in healthy subjects.

It has been discovered after thorough research of MIF and antibodies thereto that the antibodies RAB9, RAB4 and RAB0, as well as RAM9, RAM4 and RAM0, specifically bind to oxMIF (and are incapable of binding to redMIF).

In earlier experiments carried out by the inventors, it could be shown that oxidative procedures like cystine-mediated oxidation, GSSG (ox. Glutathione)-mediated oxidation or incubation of MIF with Proclin®300 or protein crosslinkers (e.g. BMOE) causes binding of MIF to the above mentioned antibodies.

Also, it was previously found that
Redox modulation (Cys/Glu-mediated mild oxidation) of recombinant MIF (human, murine, rat, CHO, monkey)) or treatment of recombinant MIF with Proclin®300 or protein crosslinkers leads to the binding of Baxter's anti-MIF antibodies RAB9, RAB4 and RAB0;
Reduction of oxMIF leads to the loss of Ab binding;
Specificity for oxMIF-isoforms correlates with biological Ab efficacy (especially in vivo); and that
oxMIF levels can be correlated with a disease state.

This additional knowledge regarding (ox)MIF served as a basis for the further studies of the present inventors. Thus, preferred embodiments of the present invention are:
1. An anti-MIF antibody for use in the treatment of cancer in a human patient, wherein the cancer contains mutant TP53 and/or mutant RAS.
2. The anti-MIF antibody according to item 1 for the use according to item 1, wherein the cancer contains mutant TP53 but not mutant RAS.
3. The anti-MIF antibody according to item 1 for the use according to item 1, wherein the cancer contains mutant RAS but not mutant TP53.
4. The anti-MIF antibody according to any of the preceding items for the use according to any of the preceding items, wherein the anti-MIF antibody is to be used in combination with a chemotherapeutic agent, which is preferably gemcitabine, mitoxantrone, cisplatin, capecitabine, 5-fluorouracil, leucovorin and/or doxorubicin.
5. The anti-MIF antibody according to items 1 or 4 for the use according to items 1 or 4, wherein the cancer contains mutant TP53 and mutant RAS.
6. The anti-MIF antibody according to any of the preceding items for the use according to any of the preceding items, wherein the cancer is selected from the following group: pancreatic cancer, ovarian cancer, prostate cancer, breast cancer, colorectal cancer, lung cancer and colon cancer, more preferred pancreatic cancer, colorectal cancer, prostate cancer and ovarian cancer.
7. The anti-MIF antibody according to item 4 for the use according to item 4, wherein the cancer is pancreatic cancer, preferably pancreatic carcinoma.
8. The anti-MIF antibody according to any of the preceding items for the use according to any of the preceding items, wherein the use is a use for treating effects caused by mutant RAS.
9. The anti-MIF antibody according to item 8 for the use according to item 8, wherein the effect caused by mutant RAS is cancer-induced inflammatory environment.
10. The anti-MIF antibody according to item 8 for the use according to item 8, wherein the effect caused by mutant RAS is angiogenesis.
11. The anti-MIF antibody according to item 8 for the use according to item 8, wherein the effect caused by mutant RAS is cancer cell proliferation.
12. The anti-MIF antibody according to item 11 for the use according to item 11, wherein the cancer cell proliferation is reduced through an induction of cancer cell apoptosis.
13. The anti-MIF antibody according to item 8 for the use according to item 8, wherein the effect caused by mutant RAS is cancer metastasis.
14. The anti-MIF antibody according to any of the preceding items for the use according to any of the preceding items, wherein the anti-MIF antibody is selected from the following group: anti-MIF antibody RAM9, RAM0 and/or RAM4.
15. The anti-MIF antibody according to any of items 4 to 14 for the use according to any of items 4 to 14, wherein the chemotherapeutic agent is gemcitabine.
16. The anti-MIF antibody according to any of items 4 to 6 and 8 to 14 for the use according to any of items 4 to 6 and 8 to 14, wherein the cancer is metastatic colorectal cancer containing mutant RAS, wherein the chemotherapeutic agent is 5-fluorouracil and leucovorin, wherein the anti-MIF antibody is preferably RAM9, and wherein the treatment is a third-line therapy.
17. The anti-MIF antibody anti-MIF antibody according to any of items 4 to 6 and 8 to 14 for the use according to any of items 4 to 6 and 8 to 14, wherein the anti-MIF antibody is RAM9, the chemotherapeutic agent is doxorubicin, optionally in combination with cisplatin, and the cancer is ovarian cancer.
18. The anti-MIF antibody anti-MIF antibody according to any of items 4 to 6 and 8 to 14 for the use according to any of items 4 to 6 and 8 to 14, wherein the anti-MIF antibody is RAM9, the chemotherapeutic agent is gemcitabine and the cancer is pancreas carcinoma.
19. The anti-MIF antibody according to any of items 4 to 6 and 8 to 14 for the use according to any of items 4 to 6 and 8 to 14, wherein the anti-MIF antibody is RAM0, the chemotherapeutic agent is doxorubicin, optionally in combination with cisplatin and the cancer is ovarian cancer.
20. The anti-MIF antibody according to any of items 4 to 6 and 8 to 14 for the use according to any of items 4 to 6 and 8 to 14, wherein the cancer is non-small cell lung cancer, and wherein the chemotherapeutic agent is docetaxel.
21. The anti-MIF antibody according to any of items 4 to 6 and 8 to 14 for the use according to any of items 4 to 6 and 8 to 14, wherein the cancer is metastatic colorectal cancer, wherein the chemotherapeutic agent is leucovorin, oxaliplatin and 5-fluorouracil, and wherein the treatment is a first-line therapy.
22. The anti-MIF antibody according to any of items 4 to 6 and 8 to 14 for the use according to any of items 4 to 6 and 8 to 14, wherein the cancer is metastatic colorectal cancer containing mutant RAS, and wherein the chemotherapeutic agent is capecitabine.
23. The anti-MIF antibody according to any of items 4 to 6 and 8 to 14 for the use according to any of items 4 to 6 and 8 to 14, wherein the anti-MIF antibody is RAM0, the chemotherapeutic agent is gemcitabine and the cancer is pancreas carcinoma.
24. The anti-MIF antibody according to any of items 4 to 6 and 8 to 14 for the use according to any of items 4 to 6 and 8 to 14, wherein the chemotherapeutic agent is doxorubicin, optionally in combination with cisplatin and the cancer is ovarian cancer.
25. The anti-MIF antibody according to any of items 4 to 6 and 8 to 14 for the use according to any of items 4 to 6 and 8 to 14, wherein the anti-MIF antibody is RAM4, the chemotherapeutic agent is gemcitabine, and the cancer is pancreas carcinoma.
26. The anti-MIF antibody according to any of items 4 to 6 and 8 to 14 for the use according to any of items 4 to 6 and 8 to 14, wherein the anti-MIF antibody is RAM0, the chemotherapeutic agent is mitoxantrone, and the cancer is prostate cancer.
27. The anti-MIF antibody according to any of items 1 or 3-26 for the use of any of items 1 or 3-26, wherein the cancer contains mutant KRAS.

The above-mentioned antibodies are characterized and supported by both their sequences as well as by deposits as plasmids in E. coli, comprising either the light or the heavy chain of each of the above mentioned antibodies RAB0, RAB4 and RAB9, respectively, as well as RAM0, RAM4 and RAM9, respectively.

The plasmids are characterized by their DSM number which is the official number as obtained upon deposit under the Budapest Treaty with the German Collection of Microorganisms and Cell Cultures (DSMZ), Mascheroder Weg 1 b, Braunschweig, Germany. The plasmids were deposited in E. coli strains, respectively. The plasmid with the DSM 25110 number comprises the light chain sequence of the anti-MIF antibody RAB4. The plasmid with the DSM 25112 number comprises the heavy chain (IgG4) sequence of the anti-MIF antibody RAB4.

The co-expression of plasmids DSM 25110 and DSM 25112 in a suitable host cell results in the production of preferred anti-MIF antibody RAB4.

The plasmid with the DSM 25111 number comprises the light chain sequence of the anti-MIF antibody RAB9. The plasmid with the DSM 25113 number comprises the heavy chain (IgG4) sequence of the anti-MIF antibody RAB9.

The co-expression of plasmids DSM 25111 and DSM 25113 in a suitable host cell results in the production of preferred anti-MIF antibody RAB9.

The plasmid with the DSM 25114 number comprises the light chain sequence of the anti-MIF antibody RAB0. The plasmid with the DSM 25115 number comprises the heavy chain (IgG4) sequence of the anti-MIF antibody RAB0.

The co-expression of plasmids DSM 25114 and DSM 25115 in a suitable host cell results in the production of preferred anti-MIF antibody RAB0.

Similarly, the light and heavy chains of RAM0, RAM9 and RAM4 have been similarly deposited under the Budapest Treaty on April 12, 2012 with the DSMZ, Braunschweig, Germany. The following designations have been used:

RAM9—heavy chain: E. coli GA.662-01.pRAM9hc—DSM 25860.

RAM4—light chain: E. coli GA.906-04.pRAM4lc—DSM 25861.

RAM9—light chain: E. coli GA.661-01.pRAM9lc—DSM 25859.

RAM4—heavy chain: E. coli GA.657-02-pRAM4hc—DSM 25862.

RAM0—light chain: E. coli GA.906-01.pRAM0lc—DSM 25863.

RAM0—heavy chain: E. coli GA.784-01.pRAM0hc—DSM 25864.

The term “prophylactic” or “therapeutic” treatment is art-recognized and refers to administration of a drug to a subject. If it is administered prior to emergence of the unwanted condition (e.g., disease or other unwanted state of the subject) then the treatment is prophylactic, i.e., it protects the subject against developing the unwanted condition, whereas if administered after emergence of the unwanted condition, the treatment is therapeutic (i.e., it is intended to diminish, ameliorate or maintain the existing unwanted condition or side effects therefrom).

The cancer treatment according to the invention is therapeutic. The therapeutic cancer treatment according to the invention includes treatments to diminish, ameliorate or maintain the cancer. That is, the cancer therapeutic treatment according to the invention also includes maintenance therapy. It also includes palliative treatment in accordance with the meaning of the term “palliative treatment” as known in the art.

The terms “treatment that maintain(s)” or “maintenance therapy” as used herein are to be understood in accordance with the common meaning of the term “maintenance therapy” as known in the art.

The cancer treatment according to the invention can be a first-line therapy, a second-line therapy or a third-line therapy or beyond. The meaning of these first-, second- or third-line therapies is in accordance with the terminology that is commonly used by the US National Cancer Institute. Thus, a “first-line therapy” that is given to the human subject is the first treatment for the cancer. A “second-line therapy” that is given to the human subject is a treatment that is given when the initial treatment (first-line therapy) does not work, or stops working. A “third-line therapy” that is given to the human subject is a treatment that is given when both initial treatment (first-line therapy) and subsequent treatment (second-line therapy) do not work, or stop working. The distinction of whether or not a treatment is working is made based on the RECIST criteria for the evaluation of a treatment response of solid tumors and/or clinical symptoms and signs of cancer progression. These criteria are known to the person skilled in the art and have been published in Eisenhauer et al., European Journal of Cancer 45 (2009), 228-247. In a preferred embodiment, the treatment according to the invention is a second-line therapy or a third-line therapy. In a more preferred embodiment, the treatment according to the invention is a third-line therapy.

As used herein an anti-(ox)MIF compound refers to any agent that attenuates, inhibits, opposes, counteracts, or decreases the biological activity of (ox)MIF. An anti(ox)MIF compound may be an agent that inhibits or neutralizes (ox)MIF activity, for example an antibody, particularly preferred, the antibodies as described herein, even more preferred the antibodies RAB9, RAB4 and/or RAB0. Very preferred antibodies are RAM9, RAM4 and/or RAM0.

The preferred MIF antagonist in accordance with the present invention is an anti-MIF antibody. Even more preferred the anti-MIF antibody is an antibody against oxMIF. In other embodiments, the anti-oxMIF antibodies, e.g., the antibodies mentioned above or an antigen-binding portion thereof bind oxMIF with a K_{D} of less than 100 nM, preferably a K_{D} of less than 50 nM, even more preferred with a K_{D} of less than 10nM. Very preferred, the antibodies bind to oxMIF with a K_{D} of less than 5 nM.

The invention further relates to kits comprising an anti-MIF antibody or an antigen-binding portion thereof as well as preferably also a chemotherapeutic agent according to the invention. A kit may include in addition to the antibody and the optional chemotherapeutic agent, further therapeutic agents and uses thereof. A kit also can include instructions for use in a therapeutic method.

Earlier results have shown that an anti-MIF antibody that only binds oxMIF and does not bind redMIF and further inhibits GOO and/or cell proliferation induces a beneficial effect in an animal model.

### DETAILED DESCRIPTION OF THE INVENTION

The invention is further described in the figures as enclosed.

### DESCRIPTION OF THE FIGURES

FIG. 1: Anti-oxMIF inhibits phosphorylation of ERK and AKT in vitro. In order to investigate the effects of anti-oxMIF antibodies in TP53 and KRAS mutant cells, starved PC3 cells (containing a mutant KRAS gene encoding a K-ras G12V mutation, and containing a deletion mutation in the TP53 gene) were incubated in the presence of 10% FCS, 10 nM recombinant MIF, 100 nM RAM4, RAM9 or RAM0 or isotype control antibody as indicated. Cell lysates were separated by SDS-PAGE and blotted on nitrocellulose membranes, and the phosphorylated forms of ERK1/2 (FIG. 1A) or AKT (FIG. 1B) were visualized with phosphor-specific antisera. The total levels of the enzymes were determined by using antisera that recognized all forms of the enzymes and were visualized as a loading control. The figure shows the resulting western blot images.

FIG. 2: Tumor measurements in a xenograft model of ovarian cancer based on inhibition of IGROV-1 cells stably expressing the luciferase reporter gene. IGROV-1 human ovarian cancer cells (containing a wild-type KRAS gene, and containing a mutant TP53 gene encoding a p53 Y126C mutation) stably expressing the luciferase reporter gene were implanted on day 0. Four Weeks after injection of the tumor cells, the treatment was initiated by injection of 60 mg/kg RAM9, 60 mg/kg RAM0, control IgG or PBS (FIG. 2A). The treatment was carried out every other day. In the 4th_{,} 5th and 6^{th} week after injection of the tumor cells, the tumors were assessed by measuring luciferase activity (total flux in photons per second, p/s) (FIG. 2B).

FIG. 3: Tumor volume (FIG. 3D) and intratumoral levels of the cytokines, II-6 (FIG. 3B), II-8 (FIG. 3A) and GRO-alpha (FIG. 3C) were analyzed for tumors from a PC3 prostate cancer xenograft in vivo model after treatment with the indicated antibodies. The data shown represent the mean.

### DEFINITIONS AND GENERAL TECHNIQUES

Unless otherwise defined herein, scientific and technical terms used in connection with the present invention shall have the meanings that are commonly understood by those of ordinary skill in the art. Generally, nomenclatures used in connection with, and techniques of, cell and tissue culture, molecular biology, immunology, microbiology, genetics and protein and nucleic acid chemistry described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2nd ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. “MIF” or “macrophage migration inhibitory factor” refers to the protein, which is known as a critical mediator in the immune and inflammatory response, and as a counterregulator of glucocorticoids. MIF includes mammalian MIF, specifically human MIF (Swiss-Prot primary accession number: P14174), wherein the monomeric form is encoded as a 115 amino acid protein but is produced as a 114 amino acid protein due to cleavage of the initial methionine. “MIF” also includes “GIF” (glycosylation-inhibiting factor) and other forms of MIF such as fusion proteins of MIF. The numbering of the amino acids of MIF starts with the N-terminal methionine (amino acid 1) and ends with the C-terminal alanine (amino acid 115).

“oxidized MIF” or oxMIF is defined for the purposes of the invention as an isoform of MIF that occurs by treatment of MIF with mild oxidizing reagents, such as Cystine. As has been shown by the present inventors, recombinant oxMIF that has been treated this way comprises isoform(s) of MIF that share structural rearrangements with oxMIF that (e.g.) occurs in vivo after challenge of animals with bacteria. redMIF is defined for the purposes of this invention as reduced MIF and is MIF which does not bind to RAB0, RAB9 and/or RAB4.

The anti-oxMIF antibodies used in this invention are able to discriminate between ox and red MIF, which are generated by mild oxidation or reduction, respectively. The anti-oxMIF antibodies are useful to specifically detect oxMIF. Discrimination between these conformers is assessed by ELISA or surface plasmon resonance. Both techniques can be performed as well known to a person skilled in the art and as described below.

Assessing differential binding of the antibodies by Biacore™.

Binding kinetics of oxMIF and redMIF to antibody RAB9 and RAB0 are examined by surface plasmon resonance analysis using a Biacore™ 3000 System. The antibodies were coated on a CM5 (=carboxymethylated dextran) chip and recombinant MIF protein, pre-incubated with 0.2% Proclin®300, were injected. (Proclin®300 consists of oxidative isothiazolones that stabilize the oxMIF structure). In native HBS-EP buffer (=Biacore™ running buffer) without addition of ProClin®300, none of the recombinant MIF proteins bound to RAB9, RAB0 or to the reference antibody (irrelevant isotype control antibody) used as negative (background) binding control.

In a preferred embodiment, oxMIF is MIF which is differentially bound by antibody RAB9, RAB4 and/or RAB0 or an antigen-binding fragment thereof, meaning that these antibodies do bind to oxMIF while redMIF is not bound by either one of these antibodies.

In other embodiments, the anti-oxMIF antibodies, e.g., the antibodies mentioned above or an antigen-binding portion thereof bind oxMIF with a K_{D} of less than 100 nM, preferably a K_{D} of less than 50 nM, even more preferred with a K_{D} of less than 10 nM. Particularly preferred, the antibodies of the invention bind to oxMIF with a K_{D} of less than 5 nM.

The antibodies of the invention, as defined hereinabove and hereinafter, have the same specificities. They thus also show similar results in the experiments as carried out by the present inventors.

(Non-)binding of an antibody, e.g. RAB9, RAB4 or RAB0 or RAM9, RAM4 or RAM0 (to oxMIF or redMIF) can be determined as generally known to a person skilled in the art, examples being any one of the following methods: ELISA with recombinant MIF in its reduced or oxidized state, or surface plasmon resonance using recombinant MIF in its reduced or oxidized state, like the well known Biacore™ assay, described above. A preferred method for the determination of binding is surface plasmon resonance of an antibody to e.g. rec. (ox)MIF whereupon “binding” is meant to be represented by a K_{D} of less than 100 nM preferably less than 50 nM, even more preferred less than 10 nM whereas the non-binding to redMIF is characterized by a K_{D} of more than 400 nM. “Binding” and “specific binding” is used interchangeably here to denote the above. “Differential binding” in the context of this application means that a compound, in particular the antibodies as described herein, bind to oxMIF (e.g., with the K_{D} values mentioned above) while they do not bind to redMIF (with non-binding again being defined as above).

An “antibody” refers to an intact antibody or an antigen-binding portion that competes with the intact antibody for (specific) binding. See generally, Fundamental Immunology, Ch. 7 (Paul, W., ed., 2nd ed. Raven Press, N.Y. (1989)) (incorporated by reference). The term antibody includes human antibodies, mammalian antibodies, isolated antibodies and genetically engineered forms such as chimeric, camelide/camelized or humanized antibodies, though not being limited thereto.

The term “antigen-binding portion” of an antibody refers to one or more fragments of an antibody that retain the ability to specifically bind to an antigen (e.g., (ox)MIF). Antigen-binding portions may be produced by recombinant DNA techniques or by enzymatic or chemical cleavage of intact antibodies. Antigen-binding portions include e.g.—though not limited thereto—the following: Fab, Fab′, F(ab′)2, Fv, and complementarity determining region (CDR) fragments, single-chain antibodies (scFv), chimeric antibodies, antibodies and polypeptides that contain at least a portion of an antibody that is sufficient to confer specific antigen binding to the polypeptide, i.e., ox or redMIF. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia et al. J. Mol. Biol. 196:901-917 (1987), or Chothia et al., Nature 342:878-883 (1989). An antibody or antigen-binding portion thereof can be derivatized or linked to another functional molecule (e.g., another peptide or protein). For example, an antibody or antigen- binding portion thereof can be functionally linked to one or more other molecular entities, such as another antibody (e.g., a bispecific antibody or a diabody), a detectable agent, a cytotoxic agent, a pharmaceutical agent, and/or a linking molecule.

The term “KD” refers here, in accordance with the general knowledge of a person skilled in the art to the equilibrium dissociation constant of a particular antibody with the respective antigen. This equilibrium dissociation constant measures the affinity. The affinity determines how much complex is formed at equilibrium (steady state where association balances dissociation) (here: ox or redMIF and antibody).

ka=association rate constant [M-1 s-1]

kd=dissociation rate constant [s-1]

KD=equilibrium dissociation constant=kd/ka [M]

The term “human antibody” refers to any antibody in which the variable and constant domains are human sequences. The term encompasses antibodies with sequences derived from human genes, but which have been changed, e.g. to decrease possible immunogenicity, increase affinity, eliminate cysteines that might cause undesirable folding, etc. The term encompasses such antibodies produced recombinantly in non-human cells, which might e.g. impart glycosylation not typical of human cells.

The term “humanized antibody” refers to antibodies comprising human sequences and containing also non-human sequences; in particular, a “humanized antibody” refers to a non-human antibody where human sequences have been added and/or replace the non-human sequences.

The term “camelized antibody” refers to antibodies wherein the antibody structure or sequences has been changed to more closely resemble antibodies from camels, also designated camelid antibodies. Methods for the design and production of camelized antibodies are part of the general knowledge of a person skilled in the art.

The term “chimeric antibody” refers to an antibody that comprises regions from two or more different species. The term “isolated antibody” or “isolated antigen-binding portion thereof” refers to an antibody or an antigen-binding portion thereof that has been identified and selected from an antibody source such as a phage display library or a B-cell repertoire.

The production of the anti-(ox)MIF antibodies according to the present invention includes any method for the generation of recombinant DNA by genetic engineering, e.g. via reverse transcription of RNA and/or amplification of DNA and cloning into expression vectors. In some embodiments, the vector is a viral vector, wherein additional DNA segments may be ligated into the viral genome. In some embodiments, the vector is capable of autonomous replication in a host cell into which it is introduced (e.g., bacterial vectors having a bacterial origin of replication and episomal mammalian vectors). In other embodiments, the vector (e.g., non-episomal mammalian vectors) can be integrated into the genome of a host cell upon introduction into the host cell, and thereby replicated along with the host genome. Moreover, certain vectors are capable of directing the expression of genes to which they are operatively linked. Such vectors are referred to herein as “recombinant expression vectors” (or simply, “expression vectors”).

Anti-(ox)MIF antibodies can be produced inter alia by means of conventional expression vectors, such as bacterial vectors (e.g., pBR322 and its derivatives), or eukaryotic vectors. Those sequences that encode the antibody can be provided with regulatory sequences that regulate the replication, expression and/or secretion from the host cell. These regulatory sequences comprise, for instance, promoters (e.g., CMV or SV40) and signal sequences. The expression vectors can also comprise selection and amplification markers, such as the dihydrofolate reductase gene (DHFR), hygromycin-B-phosphotransferase, and thymidine-kinase. The components of the vectors used, such as selection markers, replicons, enhancers, can either be commercially obtained or prepared by means of conventional methods. The vectors can be constructed for the expression in various cell cultures, e.g., in mammalian cells such as CHO, COS, HEK293, NSO, fibroblasts, insect cells, yeast or bacteria such as E. coli. In some instances, cells are used that allow for optimal glycosylation of the expressed protein.

The anti-(ox)MIF antibody light chain gene(s) and the anti-(ox)MIF antibody heavy chain gene(s) can be inserted into separate vectors or the genes are inserted into the same expression vector. The antibody genes are inserted into the expression vector by standard methods, e.g., ligation of complementary restriction sites on the antibody gene fragment and vector, or blunt end ligation if no restriction sites are present.

The production of anti-(ox)MIF antibodies or antigen-binding fragments thereof may include any method known in the art for the introduction of recombinant DNA into eukaryotic cells by transfection, e.g. via electroporation or microinjection. For example, the recombinant expression of anti-(ox)MIF antibody can be achieved by introducing an expression plasmid containing the anti-(ox)MIF antibody encoding DNA sequence under the control of one or more regulating sequences such as a strong promoter, into a suitable host cell line, by an appropriate transfection method resulting in cells having the introduced sequences stably integrated into the genome. The lipofection method is an example of a transfection method which may be used according to the present invention.

The production of anti-(ox)MIF antibodies may also include any method known in the art for the cultivation of said transformed cells, e.g. in a continuous or batchwise manner, and the expression of the anti-(ox)MIF antibody, e.g. constitutive or upon induction. It is referred in particular to WO 2009/086920 for further reference for the production of anti-(ox)MIF antibodies. In a preferred embodiment, the anti-(ox)MIF antibodies as produced according to the present invention bind to oxMIF or an epitope thereof. Particularly preferred antibodies in accordance with the present invention are antibodies RAB9, RAB4 and/or RAB0, as well as RAM9, RAM4 and/or RAM0.

The sequences of these antibodies are partly also disclosed in WO 2009/086920; see in addition the sequence list of the present application and the following:

SEQ ID NO: 1 for the amino acid sequence of the light chain of RAB9:
DIQMTQSPSS LSASVGDRVT ITCRSSQRIM TYLNWYQQKP GKAPKLLIFV ASHSQSGVPS RFRGSGSETD
FTLTISGLQP EDSATYYCQQ SFWTPLTFGG GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC,
SEQ ID NO: 2 for the amino acid sequence of the light chain of RAB4:
DIQMTQSPGT LSLSPGERAT LSCRASQGVS SSSLAWYQQK PGQAPRLLIY GTSSRATGIP DRFSGSASGT
DFTLTISRLQ PEDFAVYYCQ QYGRSLTFGG GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC,
SEQ ID NO: 3 for the amino acid sequence of the light chain of RAB0:
DIQMTQSPGT LSLSPGERAT LSCRASQGVS SSSLAWYQQK PGQAPRLLIY GTSSRATGIP DRFSGSASGT
DFTLTISRLQ PEDFAVYYCQ QYGRSLTFGG GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC,
SEQ ID NO: 4 for the amino acid sequence of the light chain of RAB2:
DIQMTQSPVT LSLSPGERAT LSCRASQSVR SSYLAWYQQK PGQTPRLLIY GASNRATGIP DRFSGSGSGT
DFTLTISRLE PEDFAVYYCQ QYGNSLTFGG GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC,
SEQ ID NO: 5 for the amino acid sequence of the heavy chain of RAB9:
EVQLLESGGG LVQPGGSLRL SCAASGFTFS IYSMNWVRQA PGKGLEWVSS
IGSSGGTTYY ADSVKGRFTI SRDNSKNTLY LQMNSLRAED TAVYYCAGSQ
WLYGMDVWGQ GTTVTVSSAS TKGPSVFPLA PCSRSTSEST AALGCLVKDY
FPEPVTVSWN SGALTSGVHT FPAVLQSSGL YSLSSVVTVP SSSLGTKTYT
CNVDHKPSNT KVDKRVESKY GPPCPPCPAP EFLGGPSVFL FPPKPKDTLM
ISRTPEVTCV VVDVSQEDPE VQFNWYVDGV EVHNAKTKPR EEQFNSTYRV
VSVLTVLHQD WLNGKEYKCK VSNKGLPSSI EKTISKAKGQ PREPQVYTLP
PSQEEMTKNQ VSLTCLVKGF YPSDIAVEWE SNGQPENNYK TTPPVLDSDG
SFFLYSRLTV DKSRWQEGNV FSCSVMHEAL HNHYTQKSLS LSLGK,
SEQ ID NO: 6 for the amino acid sequence of the heavy chain of RAB4:
EVQLLESGGG LVQPGGSLRL SCAASGFTFS IYAMDWVRQA PGKGLEWVSG
IVPSGGFTKY ADSVKGRFTI SRDNSKNTLY LQMNSLRAED TAVYYCARVN
VIAVAGTGYY YYGMDVWGQG TTVTVSSAST KGPSVFPLAP CSRSTSESTA
ALGCLVKDYF PEPVTVSWNS GALTSGVHTF PAVLQSSGLY SLSSVVTVPS
SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE
EQFNSTYRVV SVLTVLHODW LNGKEYKCKV SNKGLPSSIE KTISKAKGQP
REPQVYTLPP SQEEMTKNQV SLTCLVKGFY PSDIAVEWES NGQPENNYKT
TPPVLDSDGS FFLYSRLTVD KSRWQEGNVF SCSVMHEALH NHYTQKSLSL
SLGK,
SEQ ID NO: 7 for the amino acid sequence of the heavy chain of RAB0:
EVQLLESGGG LVQPGGSLRL SCAASGFTFS WYAMDWVRQA PGKGLEWVSG
IYPSGGRTKY ADSVKGRFTI SRDNSKNTLY LQMNSLRAED TAVYYCARVN
VIAVAGTGYY YYGMDVWGQG TTVTVSSAST KGPSVFPLAP CSRSTSESTA
ALGCLVKDYF PEPVTVSWNS GALTSGVHTF PAVLQSSGLY SLSSVVTVPS
SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE
EQFNSTYRVV SVLTVLHQDW LNGKEYKCKV SNKGLPSSIE KTISKAKGQP
REPQVYTLPP SQEEMTKNQV SLTCLVKGFY PSDIAVEWES NGQPENNYKT
TPPVLDSDGS FFLYSRLTVD KSRWQEGNVF SCSVMHEALH NHYTQKSLSL
SLGK,
SEQ ID NO: 8 for the amino acid sequence of the heavy chain of RAB2:
EVQLLESGGG LVQPGGSLRL SCAASGFTFS IYAMDWVRQA PGKGLEWVSG
IVPSGGFTKY ADSVKGRFTI SRDNSKNTLY LQMNSLRAED TAVYYCARVN
VIAVAGTGYY YYGMDVWGQG TTVTVSSAST KGPSVFPLAP CSRSTSESTA
ALGCLVKDYF PEPVTVSWNS GALTSGVHTF PAVLQSSGLY SLSSVVTVPS
SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE
EQFNSTYRVV SVLTVLHQDW LNGKEYKCKV SNKGLPSSIE KTISKAKGQP
REPQVYTLPP SQEEMTKNQV SLTCLVKGFY PSDIAVEWES NGQPENNYKT
TPPVLDSDGS FFLYSRLTVD KSRWQEGNVF SCSVMHEALH NHYTQKSLSL
SLGK,
SEQ ID NO: 9 for the amino acid sequence of RAM0hc:
EVQLLESGGG LVQPGGSLRL SCAASGFTFS WYAMDWVRQA PGKGLEWVSG IYPSGGRTKY
ADSVKGRFTI SRDNSKNTLY LQMNSLRAED TAVYYCARVN VIAVAGTGYY YYGMDVWGQG
TTVTVSSAST KGPSVFPLAP SSKSTSGGTA ALGCLVKDYF PEPVTVSWNS GALTSGVHTF
PAVLQSSGLY SLSSVVTVPS
SSLGTQTYIC NVNHKPSNTK VDKRVEPKSC DKTHTCPPCP APELLGGPSV FLFPPKPKDT
LMISRTPEVT CVVVDVSHED PEVKFNWYVD GVEVHNAKTK PREEQYNSTY RVVSVLTVLH
QDWLNGKEYK CKVSNKALPA PIEKTISKAK GQPREPQVYT LPPSREEMTK NQVSLTCLVK
GFYPSDIAVE WESNGQPENN YKTTPPVLDS DGSFFLYSKL TVDKSRWQQG NVFSCSVMHE
ALHNHYTQKS LSLSPGK,
SEQ ID NO: 10 for the amino acid sequence of RAM0lc:
DIQMTQSPGT LSLSPGERAT LSCRASQGVS SSSLAWYQQK PGQAPRLLIY GTSSRATGIP
DRFSGSASGT DFTLTISRLQ PEDFAVYYCQ QYGRSLTFGG GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC,
SEQ ID NO: 11 for the amino acid sequence of RAM9hc:
EVQLLESGGG LVQPGGSLRL SCAASGFTFS IYSMNWVRQA PGKGLEWVSS IGSSGGTTYY
ADSVKGRFTI SRDNSKNTLY LQMNSLRAED TAVYYCAGSQ WLYGMDVWGQ GTTVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKRVEPKS CDKTHTCPPC PAPELLGGPS
VFLFPPKPKD TLMISRTPEV TCVVVDVSHE DPEVKFNWYV DGVEVHNAKT KPREEQYNST
YRVVSVLTVL HQDWLNGKEY KCKVSNKALP APIEKTISKA KGQPREPQVY TLPPSREEMT
KNQVSLTCLV KGFYPSDIAV EWESNGQPEN NYKTTPPVLD SDGSFFLYSK LTVDKSRWQQ
GNVFSCSVMH EALHNHYTQK SLSLSPGK,
SEQ ID NO: 12 for the amino acid sequence of RAM9lc:
DIQMTQSPSS LSASVGDRVT ITCRSSQRIM TYLNWYQQKP GKAPKLLIFV ASHSQSGVPS
RFRGSGSETD FTLTISGLQP EDSATYYCQQ SFWTPLTFGG GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC,
SEQ ID NO: 13 for the amino acid sequence of RAM4hc:
EVQLLESGGG LVQPGGSLRL SCAASGFTFS IYAMDWVRQA PGKGLEWVSG IVPSGGFTKY
ADSVKGRFTI SRDNSKNTLY LQMNSLRAED TAVYYCARVN VIAVAGTGYY YYGMDVWGQG
TTVTVSSAST KGPSVFPLAP SSKSTSGGTA ALGCLVKDYF PEPVTVSWNS GALTSGVHTF
PAVLQSSGLY SLSSVVTVPS SSLGTQTYIC NVNHKPSNTK VDKRVEPKSC DKTHTCPPCP
APELLGGPSV FLFPPKPKDT LMISRTPEVT CVVVDVSHED PEVKFNVVWD GVEVHNAKTK
PREEQYNSTY RVVSVLTVLH QDWLNGKEYK CKVSNKALPA PIEKTISKAK GQPREPQVYT
LPPSREEMTK NQVSLTCLVK GFYPSDIAVE WESNGQPENN YKTTPPVLDS DGSFFLYSKL
TVDKSRWQQG NVFSCSVMHE ALHNHYTQKS LSLSPGK,
SEQ ID NO: 14 for the amino acid sequence of RAM4lc:
DIQMTQSPGT LSLSPGERAT LSCRASQGVS SSSLAWYQQK PGQAPRLLIY GTSSRATGIP
DRFSGSASGT DFTLTISRLQ PEDFAVYYCQ QYGRSLTFGG GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC.

The anti-MIF antibody of the invention is preferably an isolated monoclonal antibody. The anti-MIF antibody can be an IgG, an IgM, an IgE, an IgA, or an IgD molecule. In other embodiments, the anti-MIF antibody is an IgG1, IgG2, IgG3 or IgG4 subclass. In other embodiments, the antibody is either subclass IgG1 or IgG4. In other embodiments, the antibody is subclass IgG4. In some embodiments, the IgG4 antibody has a single mutation changing the serine (serine228, according to the Kabat numbering scheme) to proline. Accordingly, the CPSC sub-sequence in the Fc region of IgG4 becomes CPPC, which is a sub-sequence in IgG1 (Angel et al. Mol Immunol. 1993, 30, 105-108).

Additionally, the production of anti-(ox)MIF antibodies may include any method known in the art for the purification of an antibody, e.g., via anion exchange chromatography or affinity chromatography. In one embodiment the anti-(ox)MIF antibody can be purified from cell culture supernatants by size exclusion chromatography.

The terms “center region” and “C-terminal region” of MIF refer to the region of human MIF comprising amino acids 35-68 and aa 86-115, respectively, preferably aa 50-68 and aa 86 to 102 of human MIF, respectively. Particularly preferred antibodies of the present invention bind to either region aa 50-68 or region aa 86-102 of human MIF. This is also reflected by the preferred antibodies of the invention, like RAB0, RAB4 RAB2 and RAB9 as well as RAM4, RAM9 and RAM0 which bind as follows:

RAB4 and RAM4: aa 86-102

RAB9 and RAM9: aa 50-68

RAB0 and RAM0: aa 86-102

RAB2: aa 86-102

The term “epitope” includes any protein determinant capable of specific binding to an immunoglobulin or an antibody fragment. Epitopic determinants usually consist of chemically active surface groupings of molecules such as exposed amino acids, amino sugars, or other carbohydrate side chains and usually have specific three-dimensional structural characteristics, as well as specific charge characteristics.

The term “vector” refers to a nucleic acid molecule capable of transporting another nucleic acid to which it has been linked. In some embodiments, the vector is a plasmid, i.e., a circular double stranded DNA loop into which additional DNA segments may be ligated.

The term “host cell” refers to a cell line, which is able to produce a recombinant protein after introducing an expression vector. The term “recombinant cell line”, refers to a cell line into which a recombinant expression vector has been introduced. It should be understood that “recombinant cell line” means not only the particular subject cell line but also the progeny of such a cell line. Because certain modifications may occur in succeeding generations due to either mutation or environmental influences, such progeny may not, in fact, be identical to the parent cell, but are still included within the scope of the term “recombinant cell line” as used herein.

The host cell type according to the present invention is e.g. a COS cell, CHO cell or, e.g., an HEK293 cell, or any other host cell known to a person skilled in the art, thus also for example including bacterial cells, like e.g. E. coli cells. In one embodiment, the anti-MIF antibody is expressed in a DHFR-deficient CHO cell line, e.g., DX611, and with the addition of G418 as a selection marker. When recombinant expression vectors encoding antibody genes are introduced into CHO host cells, the antibodies are produced by culturing the host cells for a period of time sufficient to allow for expression of the antibody in the host cells or secretion of the antibody into the culture medium in which the host cells are grown.

Anti-(ox)MIF antibodies can be recovered from the culture medium using standard protein purification methods.

A second active ingredient of the combination therapy, which is a preferred embodiment of the present invention, is a chemotherapeutic.

Chemotherapeutic agents in the general sense thereof, are compounds, which can be used for the treatment of a disease or disorder that arises from bacterial, viral or parasitic infection or that is due to transformation of normal cells (cancer). One particular indication of chemotherapy is cancer. Chemotherapeutic agents can act for example by killing cells that divide more rapidly than other cells, and thus target cancer cells which commonly divide more rapidly than non-cancerous cells. Most chemotherapeutic agents work by impairing cell division at one of several stages of the cell cycle. Thus, they are able to target those cells that divide more rapidly. Chemotherapeutic agents can be either cytostatic, i.e., they slow down or abrogate the growth or division of cells; other chemotherapeutic agents can cause damage to cells and kill them; in that case they are termed cytotoxic. Most cytotoxic drugs inflict a damage that per se does not suffice to kill a cell but that generates a stimulus to initiate programmed cell death (apoptosis).

In general, major classes of chemotherapeutic drugs are alkylating agents, anti-metabolites, anthracyclines, plant alkaloids, topoisomerase inhibitors and other anti-tumour agents. Most commonly, as mentioned above, these drugs affect one or several stages of the cell cycle; they can also affect DNA synthesis or DNA integrity. Other chemotherapeutics do not directly interfere with DNA. These are newer classes of chemotherapeutics and can include monoclonal antibodies and tyrosine kinase inhibitors like imatinib mesylate. Other examples are chemotherapeutic hormones and hormone antagonists, e.g. glucocorticosteroids.

Examples for alkylating agents, which alkylate nucleophilic functional groups are mechlorethamine, cyclophosphamide, chlorambucil, melphalane, trofosfamide, ifosfamide, carmustine, lomustine, dacarbazine, temozolomide, mitomycine C and many others. Cisplatin, carboplatin, oxaliplatin and other platinum containing compounds form stable complexes with DNA.

Cytotoxic anti-metabolites are folic acid analogues (e.g., methotrexate/aminopterin, raltitrexed, pemetrexed, or leucovorin (also termed folinic acid)), purine analogs (e.g., 6-mercaptopurine, azathioprine, thioguanine, fludarabine, cladribine) or pyrimidine analogs (cytarabine, gemcitabine, deazacytidine, 5-fluorouracil and its prodrugs including capecitabine). Antimetabolites either inhibit DNA-synthesis by interfering with crucial steps in the de novo synthesis of purine and pyrimidine nucleotides or they become incorporated into DNA during the S-phase of the cell cycle, where they interfere with DNA-folding, DNA-repair or methylation. Alternatively, some compounds also become incorporated into RNA.

Examples for alkaloids and terpenoids which are derived from plants and block cell division by preventing microtubule function are vinca-alkaloids and taxanes. Particularly well known vinca-alkaloids are vincristine, vinblastine, vinorelbine and vindesine. Podophyllotoxin is an additional example of a plant-derived compound. An example for a taxane is docetaxel or paclitaxel. Another example is abraxane, an albumin bound paclitaxel. Estramustin is an example of a synthetic compound that targets tubulin.

Examples of topoisomerase inhibitors, which are inhibitors of enzymes that maintain the topology of DNA, include camphtotecines like irinotecan and topotecan (type 1 topoisomerase inhibitors) or amsacrin, etoposide, etoposide phosphate and teniposide (topoisomerase-type 2 inhibitors).

Finally, examples of antineoplastic intercalating agents include dactinomycin, doxorubicin, epirubicin, bleomycin and others.

A comprehensive overview is comprised in Goodman and Gilman, The Pharmacological Basis of Therapeutics, 12th Edition, “General Principles of Cancer Chemotherapy”.

The Following are Examples for Alkylating Agents:

Mechlorethamine

Cyclophosphamide Ifosfamide

Melphalan

Chlorambucil

Procarbazine (N-methylhydrazine, MIH)

Busulfan

Camustine (BCNU)

Streptozocin

(streptozotocin)

Bendamustine

Dacarbazine (DTIC; dimethyltriazenol midazole carboxamide)

Temozolomide

Cisplatin, carboplatin, oxaliplatin

Antimetabolites are exemplary represented by

Methotrexate (Amethopterin)

Pemetrexed

Fluorouracil (5-fluorouracil; 5-FU), capecitabine

Cytarabine (cytosine arabinoside)

Gemcitabine

5-aza-cytidine

Deoxy-5-aza-cytidine

Mercaptopurine (6-mercaptopurine; 6-MP)

Pentostatin (2′-deoxycoformycin)

Fludarabine

Clofarabine

Nelarabine

while Natural Products can be selected from:

Vinblastine

Vinorelbine

Vincristine

Paclitaxel, docetaxel

Etoposide

Teniposide

Topotecan

Irinotecan

Dactinomycin

(actinomycin D)

Daunorubicin

(daunomycin, rubidomycin)

Doxorubicin

Yondelis

Mitoxantrone

Bleomycin

Mitomycin C

L-Asparaginase

Examples for Hormones and Antagonists are:

Mitotane (o.p′DDD)

Prednisone

Hydroxyprogesterone caproate,

medroxyprogesterone acetate, megestrol acetate

Dietyhlstilbestrol, ethinyl estradiol

Tamoxifen, toremifene

Anastrozole, letrozole, exemestane

Testosterone propionate, fluoxymesterone

Flutamide, casodex

Leuprolide

while examples for further agents are:

Hydroxyurea

Tretinoin, arsenic trioxide

Histone deacetylase inhibitor (vorinostat)

Imatinib

Dasatinib, nilotinib

Gefitinib, erlotinib

Sorafenib

Sunitinib

Lapatinib

Bortezomib

Interferon-alfa,

Interleukin-2

Thalidomide

Lenalidomide

Temsirolimus,

Everolimus

Chemotherapeutics have been shown to be successful in alleviation and treatment of cancer. However, most chemotherapeutics are associated with a range of side effects which are in some cases extreme, to the extent that the treatment has to be abrogated. In any case, the side effects place a further burden on the physical and mental health of a patient and should thus be avoided as far as possible.

In accordance with a preferred embodiment of the present invention, by combining a chemotherapeutic with an anti-MIF antibody, it is possible to reduce the amount of the chemotherapeutic agent which is necessary for a given treatment compared to a situation where the chemotherapeutic agent is given as the sole active ingredient. A further possibility enabled by the present invention is to maintain the dose of the chemotherapeutic as compared to the chemotherapeutic given alone and have a much higher treatment response in the patient.

This increase of the treatment response in the patient also indicates the possibility to achieve a treatment response as with a chemotherapeutic alone, with a combination of anti-MIF antibody with a lower dose of chemotherapeutic agent, e.g. in cases where the side effects of the chemotherapeutic do not allow continuous treatment with the higher dose.

It was shown quite surprisingly by the present inventors that the effect obtained by combining a chemotherapeutic with an anti-MIF antibody for the treatment of a cancer containing mutant RAS and/or mutant TP53 showed a much higher treatment response than with either the anti-MIF antibody or the chemotherapeutic agent alone.

A treatment response can easily be determined by a person skilled in the art and refers to diminishing or ameliorating or alleviating a given condition. Methods to determine such a treatment response are well known and can be for example determination of the likelihood or length of survival of a subject having a disease and being treated with a combination of MIF antagonist and chemotherapeutic agent with the likelihood or length of survival in other subjects having the same disease and being treated with either agent alone, or by determining the change of symptoms within one and the same patient over a period of time. An example well known to a person skilled in the art is the Kaplan-Meier-Plot.

Other methods/assays are well known and can be derived for example from general textbooks, like The Pharmacological Basis of Therapeutics, 12th Edition, “General Principles of Cancer Chemotherapy. Introduction”. This reference is incorporated hereby in its entirety by reference; additional methods/assays are those as described in the present examples.

Preferred chemotherapeutics used according to the present invention are gemcitabine, mitoxantrone, cisplatin, capecitabine, 5-fluorouracil, leucovorin and/or doxorubicin. Doxorubicin can be used in a preferred embodiment in combination with cisplatin. 5-fluorouracil can be used in a preferred embodiment in combination with leucovorin. Also preferred is e.g. abraxane. A preferred chemotherapeutic treatment regime is a combination therapy using leucovorin, oxaliplatin and 5-fluorouracil, which is also known as FOLFOX. Particularly preferred combinations are
a treatment of ovarian cancer with doxorubicin, preferably in combination with cisplatin, together with an anti-MIF antibody, or
a treatment of non-small cell lung cancer with docetaxel together with an anti-MIF antibody, or
a treatment of pancreatic carcinoma with gemcitabine and/or abraxane, together with an anti-MIF antibody,
a treatment of metastatic colorectal cancer (also known as mCRC) with FOLFOX together with an anti-MIF antibody, wherein the treatment is preferably a first-line therapy, or
a treatment of metastatic colorectal cancer (also known as mCRC) containing mutant RAS with capecitabine, together with an anti-MIF antibody, or
a treatment of metastatic colorectal cancer (also known as mCRC) containing mutant RAS with 5-fluorouracil and leucovorin, together with an anti-MIF antibody, wherein the treatment is preferably a third-line therapy or beyond.

In a preferred embodiment of the above combinations the anti-MIF antibody is selected from the group of RAM9, RAM4 and RAM0. In a very preferred embodiment of the above combinations the anti-MIF antibody is RAM9.

“Cancer” in the present context encompasses all disorders or diseases in which a cell or a group of cells displays uncontrolled growth, invasion (intrusion and destruction of adjacent tissues) and sometimes metastasis.

Further, in a preferred embodiment, the cancer can be MIF-related. MIF-related cancers are e.g. lymphoma, sarcoma, prostate cancer, colorectal cancer (also known as CRC) including metastatic colorectal cancer (also known as mCRC) and colon cancer, bladder cancer, ovarian cancer, melanoma, hepatocellular carcinoma, ovarian cancer, breast cancer, lung cancer including non-small cell lung cancer (NSCLC), and pancreatic cancer including pancreatic carcinoma, as well as endometriosis. Most preferably, the cancer is ovarian cancer or colorectal cancer.

The administration can principally be by all known routes. Preferred forms of administration are parenteral and intravenous (i.v.) application, most preferably parenteral application.

Further possible dosage forms which are also envisaged by the present application are dosage forms for oral administration such as tablets, capsules, sachets or pills. The granules can be used as such as a preferred dosage form, can be filled into capsules or sachets or can be further compressed into tablets or pills. Further dosage forms which are also encompassed by the present application are drinks or syrups, elixirs, tinctures, suspensions, solutions, hydrogels, films, lozenges, chewing gums, orally disintegrating tablets, mouth-washes, toothpaste, lip balms, medicated shampoos, nanosphere suspensions and microsphere tablets, as well as aerosols, inhalers, nebulisers, smoking or freebase powder forms and dosage forms for topical application like creams, gels, liniments or balms, lotions, ointments, ear drops, eye drops and skin patches.

Further encompassed are suppositories which can be used e.g. rectally or vaginally. All these dosage forms are well-known to a person skilled in the art.

Dosage forms in accordance with the present invention are oral forms like granules, coated granules, tablets, enteric coated tablets, pellets, suppositories and emulsions. Even more preferred are granules and tablets. Other dosage forms are topical dosage forms. A particular preferred administration route for the anti MIF antibody is a parenteral or intravenous application, most preferably parenteral application. A preferred administration route for the chemotherapeutic agent is oral application (e.g., a granule, liquid, sachet or tablet). A further preferred application form for the chemotherapeutic is topical application, wherein a topical application can encompass an application to the skin and/or a spray, like a nasal spray or inhaler. A further preferred administration route for a chemotherapeutic is an intravenous application or an application via a subcutaneous injection (including slow release formulations).

The term “combination” or “combination therapy” are used interchangeably here. They refer to a dosing regimen where the anti-MIF antibody is administered together with or sequentially to the chemotherapeutic or vice versa. The dosing regimen would be typically daily for chemotherapeutics and every 2 weeks for the anti-MIF antibody.

The term “half-life” as referred to herein refers to the time that a substance (e.g. an anti-MIF antibody as used according to the invention, or a chemotherapeutic as optionally also used according to the invention) needs in order to lose half of its biological activity in the respective subject (e.g. in rodents such as mice and primates such as monkeys or humans). The half-life can for instance be measured by determining plasma concentrations of the respective substance in the subject by appropriate assays known in the art or described herein. Some substances, which in particular include anti-MIF antibodies, exhibit a biphasic elimination kinetics with an initial fast elimination phase for the substance and a subsequent slower elimination phase for the substance. For such substances such as anti-MIF antibodies, an initial (shorter) half-life and a subsequent terminal (longer) half-life can be determined. The terminal half-life of anti-MIF antibodies in primates is about 5 to 10 days.

The term “wherein the cancer contains mutant TP53” refers to a cancer that contains an inactivating mutation present in the TP53 gene.

Inactivating mutations in the TP53 gene are mutations that inactivate the tumor suppressor functions of the p53 protein. Such mutations are well known to the person skilled in the art. These mutations particularly include (in order of decreasing frequency) missense substitutions, frameshift insertions and deletions, and nonsense mutations (Olivier et al., Hum Mutat. 2002, 19(6), 607-14). The most common TP53 mutations that impair the tumor suppressor functions of the p53 protein are missense mutations. Frequent missense mutations include, but are not limited to, mutations that are found in codons of the TP53 gene that correspond to p53 residues Y126 (such as Y126C), V143 (such as V143A), R172 (such as R172H), C174 (such as C174Y), R175 (such as R175H), H179 (such as H179E), L194 (such as L194F), R213 (such as R2130), Y220 (such as Y220C), G245 (such as G245S), R248 (such as R248W or R2480), R249 (such as R249S), R273 (such as R273H), R280 (such as R280K), D281 (such as D281G), and R282 (such as R282W). Most

TP53 missense mutations are found in one TP53 allele in connection with a loss of heterozygosity of the remaining wild-type p53 allele.

TP53 mutations can be detected by any suitable methods. Such methods are well-known in the art and include validated diagnostic tests which are commercially available. Such methods also include known polymerase chain reaction (PCR) techniques using genomic cancer DNA as a template, e.g. PCR methods using TP53 mutation-specific sets of primers (such as primers with a perfect nucleotide complementarity to the respective mutations and imperfect complementarity to the wild-type TP53 sequence) or TP53 mutation-specific sets of PCR probes (such as probes with a perfect nucleotide complementarity to the mutation and imperfect complementarity to the wild-type TP53 sequence). Such methods also include any methods that can be used for the sequencing of TP53 sequences that contain the mutations, e.g. methods that amplify single exons out of the eleven TP53 exons or sequential parts of these exons (e.g. by PCR amplification), followed by purification and sequencing of the amplified sequences. Sequencing can be performed by any suitable methods known in the art, e.g. based on the Sanger method (e.g. by using a 16-capillary automated sequencer such as ABI PRISM® 3100 Genetic Analyzer, Applied Biosystems), or based on pyrosequencing or next generation sequencing methods. Preferable methods for TP53 sequencing are methods published by the International Agency for Research on Cancer (IARC) and are well known to the skilled person. Mutations in the TP53 gene that impair the tumor suppressor functions of the p53 protein can be functionally verified by assays known in the art, e.g. by p53 reporter gene assays that measure the capability of mutant p53 to regulate transcription of reporter genes by binding to its known p53 consensus sequences. Such consensus sequences and assays, respectively, are commonly used and have for instance been described by el-Deiry WS et al, Nat Genet 1, 45-49 and Li et al., Acta Biochim Biophys Sin (Shanghai). 2007, 39(3), 181-6. A p53 consensus binding site typically consists of two copies of the 10 base pair motif 5′-PuPuPuC(A/T)(T/A)GPyPyPy-3′ (Pu=purine; Py=pyrimidine) separated by 0-13 or 0-14 base pairs. Alternatively, mutations in the TP53 gene that impair the tumor suppressor functions of the p53 protein can be verified by measuring the capability of the mutant TP53 gene to express functional p53 protein, as measured by a stimulation of the expression of p53 target genes such as CDKN1A, MIR34A, PUMA, BAX, the p21(WAF1) gene, GADD45, MDM2 or DR5.

The term “wherein the cancer contains mutant RAS” refers to a cancer that contains a mutation present in the KRAS and/or in the NRAS gene.

As used herein, the terms “mutant” or “mutations” in connection with the KRAS and NRAS genes include all mutations present in these genes. Mutations in the KRAS and NRAS genes are well known to the person skilled in the art. KRAS mutations include, but are not limited to, mutations that are found in the following codons of the KRAS gene: codon 12 (such as G12V, G12D, G12C, G125, G12A, G12R), codon 13 (such as G13D, G13C, G135, G13R, G13V, G13A), codon 61 (such as Q61H, Q61L, Q61R, Q61K, Q61P, Q61E), codon 117 and codon 146 (Douillard et al., N Engl J Med. 2013, 369(11), 1023-34; Phipps et al., Br J Cancer. 2013,108(8), 1757-64). NRAS mutations include, but are not limited to, mutations that are found in the following codons of the NRAS gene: Codons 12, 13 or 61.

RAS mutations can be detected by any suitable methods. Well-known methods for the detection of these mutations include, but are not limited to known polymerase chain reaction (PCR) techniques using genomic cancer DNA as a template, e.g. PCR methods using RAS mutation-specific sets of primers (such as primers with a perfect nucleotide complementarity to the respective mutations and imperfect complementarity to the wild-type RAS sequences) or RAS mutation-specific sets of PCR probes (such as probes with a perfect nucleotide complementarity to the mutation and imperfect complementarity to the wild-type RAS sequences). Such methods also include any methods that can be used for the sequencing of RAS sequences that contain the mutations, e.g. methods that amplify single exons or sequential parts of these exons (e.g. by PCR amplification), followed by purification and sequencing of the amplified sequences. Sequencing can be performed by any suitable methods known in the art, e.g. based on the Sanger method (e.g. by using a 16-capillary automated sequencer such as ABI PRISM® 3100 Genetic Analyzer, Applied Biosystems), or based on pyrosequencing or next generation sequencing methods. Methods for RAS sequencing have been reviewed, for example, in Tan and Du, World J Gastroenterol. Oct 7, 2012; 18(37): 5171-5180.

“Effects caused by mutant RAS” are effects which are caused by the mutant RAS gene(s) through the resulting mutation in the K-ras or N-ras proteins. That means that if a RAS gene is mutated by a missense mutation as indicated above, it will be transcribed, and the resulting mRNA will be translated into a mutant K-ras or N-ras protein that causes the effects. Effects caused by mutant RAS are preferably selected from cancer-induced inflammatory environment, angiogenesis, cancer cell proliferation, and cancer metastasis. The anti-MIF antibodies, preferably in combination with a chemotherapeutic agent(s), according to the invention are advantageous in that they can be used for the treatment of a cancer-induced inflammatory environment.

“Cancer-induced inflammatory environment” as used herein means an inflammatory condition that occurs within the cancer and/or within the microenvironment of the cancer. Cancer-induced inflammatory environment is characterized by the production of specific cytokines, which include TNFα, IL-6 and IL-1 (Balkwill and Mantovani, Seminars in Cancer Biology 2012, 22(1), 33-40). The presence and the level of inflammation can therefore be assessed by measuring the amounts of the cytokines TNFα, IL-6 and/or IL-1 in tumor lysates (see, for instance, non-limiting Example 3 below). Preferably, cancer-induced inflammatory environment is assessed by measuring the amounts of the cytokines TNFα and IL-6 in tumor lysates. Such measurements of the cytokines TNFα, IL-6 and/or IL-1 can be performed by methods known in the art, e.g. by antibody-based detection methods such as enzyme-linked immunosorbent assays (ELISAs) that are specific to the respective cytokine. A use of anti-MIF antibodies for the treatment of cancer-induced inflammatory environment according to the invention is advantageous in that it reduces the levels of the cytokines TNFα and IL-6 which are produced by the microenvironment of the cancer.

The treatment with anti-MIF antibodies, preferably in combination with a chemotherapeutic agent(s), according to the invention is also advantageous in that it reduces angiogenesis.

“Angiogenesis” as used herein refers to a process whereby new blood vessels are formed at the site of a tumor formed by the cancer. Angiogenesis serves to connect the tumor to the blood circulation, or to enhance the connection of the tumor to the blood circulation. Angiogenesis can be measured by methods known in the art. Such methods have, for instance, been reviewed in Auerbach et al., Clin Chem. 2003 Jan;49(1):32-40. Preferred methods to measure angiogenesis are measurements of the pro-angiogenic factor VEGF in tumor lysates, e.g. by antibody-based methods using antibodies to VEGF such as ELISA methods. Further preferred methods are methods that directly measure angiogenesis in tumor tissue sections, for instance by immunohistochemistry, Such immunohistochemistry methods include a staining of the tumor tissue sections with fluorescein isothiocyanate (FITC)-labelled lectins such as BSL-I and BSL-B4. Such lectins bind to endothelial cells and can therefore serve to visualize the blood vessels.

The treatment with anti-MIF antibodies, preferably in combination with a chemotherapeutic agent(s), according to the invention is also advantageous in that it reduces cancer cell proliferation.

“Cancer cell proliferation” refers to any increase in the number of viable cancer cells. A reduction of cancer cell proliferation by a treatment may for instance result from a reduced rate of cancer cell division (e.g. as a result of cell-cycle arrest), or from the killing of cancer cells such as a killing by apoptosis. Cancer cell proliferation can be measured by suitable methods known in the art, including (but not limited to) visual microscopy (e.g. microscopy of cancer cells cultured in vitro), in vitro metabolic assays such as those which measure mitochondrial redox potential (e.g. MTT (3-(4,5-Dimethylthiazol-2₁1)-2,5-diphenyltetrazolium bromide) assay; Resazurin staining which is also known as Alamar Blue® assay), staining of known endogenous proliferation biomarkers (e.g. Ki-67) such as a staining of Ki-67 in tumor tissue sections, and in vitro methods measuring cellular DNA synthesis (e.g. BrdU and [3H]-Thymidine incorporation assays). A reduction of cancer cell proliferation by apoptosis can be measured by methods known in the art, for instance by measuring indicators of apoptosis such as active caspase 3 and/or caspase 7, e.g. from tumor lysates. These active caspases can be measured by methods such as specific ELISA for active caspases or by immunohistochemistry.

The treatment with anti-MIF antibodies, preferably in combination with a chemotherapeutic agent(s), according to the invention is also advantageous in that it reduces cancer metastasis.

“Cancer metastasis” refers to a spread of i) a cancer from its organ of origin to another organ that is not directly connected to said organ of origin, and/or a spread of a cancer ii) from a part of the organ of origin that originally contained the cancer to another part of the same organ that is not directly connected to said part which originally contained the cancer. A reduction of cancer metastasis can be determined by detecting metastases and counting their numbers, by methods known in the art. Appropriate methods known in the art can readily be selected by a physician depending on the particular type of cancer and the organ where metastases are suspected. Such methods include various imaging methods such as bone scintigraphy (also known as bone scan) for metastases in bone, computed tomography (CT) scan (e.g. for metastases in the brain, lungs or liver), ultrasound scans (e.g. for metastases in the liver), x-ray scans (e.g. for metastases in the lungs), positron emission tomography (PET) scans (e.g. for metastases in the colon, lymph nodes, and bones) or magnetic resonance tomography (MRT).

Preferred Dosing Regimens According to the Invention are:

As explained above, it is possible to administer the anti-MIF antibody alone as a monotherapy, or together with the chemotherapeutic agent(s) in a combination therapy, or to administer the anti-MIF antibody and the chemotherapeutic agent(s) sequentially in a combination therapy. “Together with” in this context means that not more than 10 minutes have passed between the administration of the anti-MIF antibody and the administration of the chemotherapeutic. “Sequentially” means that more than 10 minutes have passed between the administration of the anti-MIF antibody and the administration of the chemotherapeutic agent. The time period can then be more than 10 min., more than 30 minutes, more than 1 hour, more than 3 hours, more than 6 hours or more than 12 hours.

Anti-MIF antibody and chemotherapeutic agents are principally dosed in a way to ensure that both compounds are present within the body during the same time period (for a certain time span). An anti-MIF antibody has a terminal half-life of typically 5 to 10 days in primates, chemotherapeutic agents a half-life of 2-48 hours.

Therefore, the above combination therapy also explicitly encompasses a sequential dosing regime where the skilled person takes into account the well known half life of the respective chemotherapeutic drug in question and the antibody in question. In view of the fact that the antibodies according to the invention have a terminal half-life of typically 5 to 10 days in primates, administration of the antibody in question to humans could be only every 5 days, every week or every 10 days. The chemotherapeutic drug to be administered in the inventive combination therapy with such an antibody has in a typical embodiment a half-life of 2-48 h; therefore, administration of the chemotherapeutic could be every 5 hours, every 6 hours, three times a day, twice a day, once daily, once a week or once per three week cycle in a typical embodiment.

Dosing of chemotherapeutics agent, as well as the combined dosing with antibodies, according to the present invention, however, will need to be determined by the practitioner on a case-by-case basis according to the specific disorder to be treated and the particulars of the afflicted subject. The person of skill in the art is aware of the respective guidelines for a given chemotherapeutic agent.

As a general understanding in curative chemotherapy, one would wish to apply the highest tolerated dose of the chemotherapeutic to achieve the desired dose intensity. The dose is reduced only if there is toxicity (i.e., neutrophil counts <4000 (but >2500)=administer half the dose, see cisplatin or cyclophosphamide). Most chemotherapeutic agents are administered on the basis of (m)g/m² body surface. Differences in tolerance and efficacy between mouse, rat and man are typically accounted for by basing the dose on body surface.

In a particularly preferred embodiment, the active ingredient would be an ingredient which should be delivered with a controlled, e.g. a delayed release. That is, the orally administrable dosage forms of the present invention comprising such an active ingredient might be provided with a coating. Thus, in a preferred embodiment the present invention is directed to granules with coatings and in particular to granules comprising active ingredients which shall be released in a controlled manner, whereby these granules have a coating.

More preferred, this coating is pharmacologically acceptable coating and particularly preferred is an enteric coating, a prolonged release coating or a delayed release coating; all such coatings are well known to a person skilled in the art.

A subset of in vivo protective anti-MIF mAbs (e.g. RAB9, RAB4, RAB0), which are directed against the pro-inflammatory cytokine MIF (Macrophage Migration Inhibitory Factor) do not bind to unmodified MIF in its reduced state. By contrast, these mAbs were shown to be highly selective for a redox dependent MIF isoform.

A particularly preferred antibody is antibody RAB9.

Another particularly preferred antibody is antibody RAM4.

Yet another particularly preferred antibody is antibody RAM0.

A very preferred antibody is antibody RAM9.

As is shown by the present invention, the therapy proposed here is advantageous in that it can be used to treat cancers which are mutant for TP53 and/or RAS, preferably in a combination therapy with a chemotherapeutic agent(s).

The present invention will be in the following described by way of the examples, whereby the examples shall be considered by no means as limiting the present invention.

### REFERENCE EXAMPLES

A) GCO-assay for Antibody Screening:

A THP1 suspension culture is centrifuged and cells are resuspended in fresh full medium to a cell density of 10⁶ cells per ml. This culture is transferred into wells of a 96-well microplate (90 μl/well) and a potential anti-MIF antibody is added to give a final concentration of 75 μg/ml. Each antibody is tested in triplicate. After o/n incubation at 37° C. dexamethasone is added to give a concentration of 2 nM and after one hour incubation at 37° C. LPS is added (3 ng/ml final concentration). After further six hours incubation at 37° C. the supernatant is harvested and the IL-6 concentrations are determined in a commercially available ELISA. The results of the triplicates are averaged and the percentage of IL-6 secretion is determined in comparison to the control antibodies. Antibodies that result in an IL-6 secretion of less than 75% are evaluated as positive.

B) Assay for Determination of IC₅₀ Values

The experimental procedure is carried out as described for the screening assay with the exception that increasing amounts of antibody are used (typically from 1-125 nM). The resultant dose response curve is expressed as % inhibition in comparison to a negative control antibody. This curve is used for calculation of the maximum inhibitory effect of the antibody (%Inh max) and the antibody concentration that shows 50% of the maximum inhibitory effect (IC₅₀).

C) Inhibition of Cell Proliferation

Serum stimulates secretion of MIF in quiescent NIH/3T3 and MIF in turn stimulates cell proliferation. Antibodies inhibiting this endogenous MIF, therefore, decrease the proliferation of quiescent NIH/3T3 cells. The reduction of proliferation is determined by the incorporation of ³H-thymidine.

1000 NIH/3T3 cells per well are incubated in a 96 well plate over the weekend at 37° C. in medium containing 10% serum. Cells are then starved over night at 37° C. by incubation in medium containing 0.5% serum. The 0.5% medium is removed and replaced by fresh medium containing 10% serum, 75 μg/ml antibody and 5 μCi/ml of 3H-thymidine. After 16 hours incubation in a CO₂ incubator at 37° C. cells are washed twice with 150 μl of cold PBS per well. Using a multi-channel pipette 150 μl of a 5% (w/v) TCA solution per well are added and incubated for 30 minutes at 4° C. Plates are washed with 150 μl PBS. Per well 75 μl of a 0.5M NaOH solution with 0.5% SDS are added, mixed and stored at room temperature. Samples are measured in a β-counter by mixing 5 ml of Ultima Gold (Packard) and 75 μl sample solution. Each determination is done in triplicate and the values are compared with the values of the control antibody by a t-test. Antibodies that significantly reduce proliferation (P<0.05) are evaluated as positive.

D) Binding Studies: Epitope Determination of Anti-MIF Antibodies

Each peptide is diluted in coupling buffer to give a peptide concentration of typically 1 μg/ml added to microplates (NUNC Immobilizer™ Amino Plate F96 Clear) and incubated over night at 4° C. (100 μl/well). As controls recombinant full length MIF and PBS are used. The plate is washed 3 times with 200 μl PBST and antibodies (2-4 μg/ml in PBS) are added (100 μl/well) and incubated for 2 hours at room temperature with gentle shaking. The plate is washed 3 times with 200 μl PBST and detection antibody (e.g. F_{c} specific anti-human IgG/HRP labelled, Sigma) is added (100 μl/well). After incubation for 1 hour at room temperature with gentle shaking, the plate is washed 3 times with 200 μl PBST. Each well is incubated with 100 μl TMB (3,3′,5,5′-tetramethylbenzidine) solution (T-0440, Sigma) for 30 minutes in the dark. Staining reaction is stopped by adding 100 μl of 1.8 M H₂SO₄-solution per well. Samples are measured at 450 nm.

E) Affinity Determination of Fab Fragments of Anti-MIF Antibodies by Biacore™

Typically, 40 RU units of human recombinant MIF are immobilized on a sensor chip with a CM5 (=carboxymethylated dextran) matrix (Biacore™). Fab fragments are injected at a concentration range of typically 6-100 nM diluted in HBS-EP. After each cycle the chip is regenerated with 50 mM NaOH+1 M NaCl. Affinities are calculated according to the 1:1 Langmuir model.

### EXAMPLES

### Example 1

### Anti-oxMIF Inhibits Phosphorylation of ERK and AKT in vitro

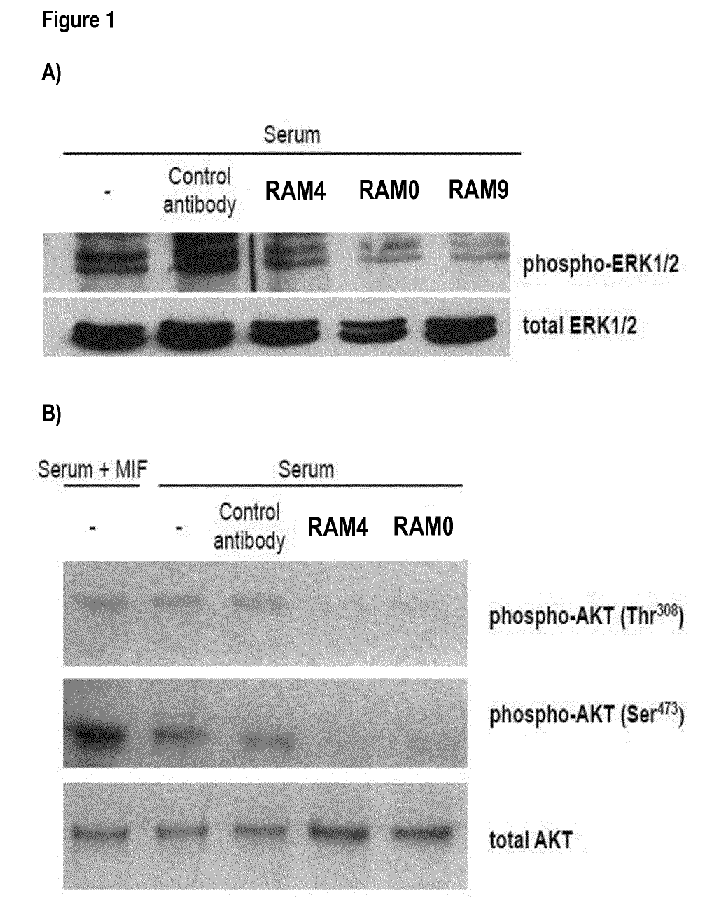

In order to investigate the effects of anti-oxMIF antibodies in TP53 and RAS mutant cells, starved PC3 cells (containing a mutant KRAS gene encoding a K-ras G12V mutation, and containing a deletion mutation in the TP53 gene) were incubated in the presence of 10% FCS, 10 nM recombinant MIF, 100 nM RAM4, RAM9 or RAM0 or isotype control antibody as indicated in FIG. 1. Cell lysates were separated by SDS-PAGE and blotted on nitrocellulose membranes, and the phosphorylated forms of ERK1/2 (FIG. 1A) or AKT (FIG. 1B) were visualized with phosphor-specific antisera. The total levels of the enzymes were determined by using antisera that recognized all forms of the enzymes and were visualized as a loading control. As can be seen from FIG. 1A and 1B, the treatment with all anti-MIF antibodies reduced the phosphorylation of ERK1/2 and AKT as compared to the untreated control or as compared to the control antibody-treated control. Mutant K-ras protein activates ERK1/2 and AKT through a cascade that leads to ERK1/2 and AKT phosphorylation. Considering the reduction of ERK1/2 and AKT phosphorylation by anti-MIF antibodies in these KRAS mutant cells that was observed in the present Example, it was concluded that anti-MIF antibodies reduce ERK1/2 and AKT phosphorylation that is stimulated by the mutant RAS gene and its mutant protein product.

### Example 2

### Tumor Measurements in a Xenograft Model of Ovarian Cancer Based on Inhibition of IGROV-1 Cells Stably Expressing the Luciferase Reporter Gene

In order to further confirm the effects of anti-MIF antibodies in the treatment of cancers containing mutations of TP53, a xenograft model of ovarian cancer based on inhibition of IGROV-1 human ovarian cancer cells (containing a wild-type KRAS gene, and containing a mutant TP53 gene encoding a p53 Y126C mutation) stably expressing the luciferase reporter gene was used. Nude mice were injected i.p. with 1×10⁷ IGROV-1 luciferase cells. Prior to start of the treatment protocol, animals were assessed for equal tumor burden and randomized into the respective treatment arms. Intraperitoneal treatment every other day started 4 weeks after cell injection/tumor establishment (10 mice per group) using 60mg/kg anti-MIF antibodies (RAM9 and RAM0), 60mg/kg control human IgG or PBSIn the 4^{th}, 5^{th} and 6^{th} week after injection of the tumor cells, the tumors were assessed by measuring luciferase activity (total flux in photons per second, p/s).

Measuring luciferase activity: At different time points following cancer cells injection or antibody treatment, tumor growth and spread were monitored by Bioluminescent Assay. Mice were injected i.p. with 150 μg/g body weight D-luciferin in PBS, and bioluminescence imaging with a charge-coupled device camera (IVIS, Xenogen, Alameda, Calif.) was initiated 10 min after injection. Bioluminescence data were analyzed using Living Image software (also from Xenogen) and presented either as relative light units (RLU) of light emission/s/cm² from ventral imaging and photon flux from a region of interest drawn over a mouse that was not given an injection of luciferin, or as total flux measurements in photons/second (p/s).

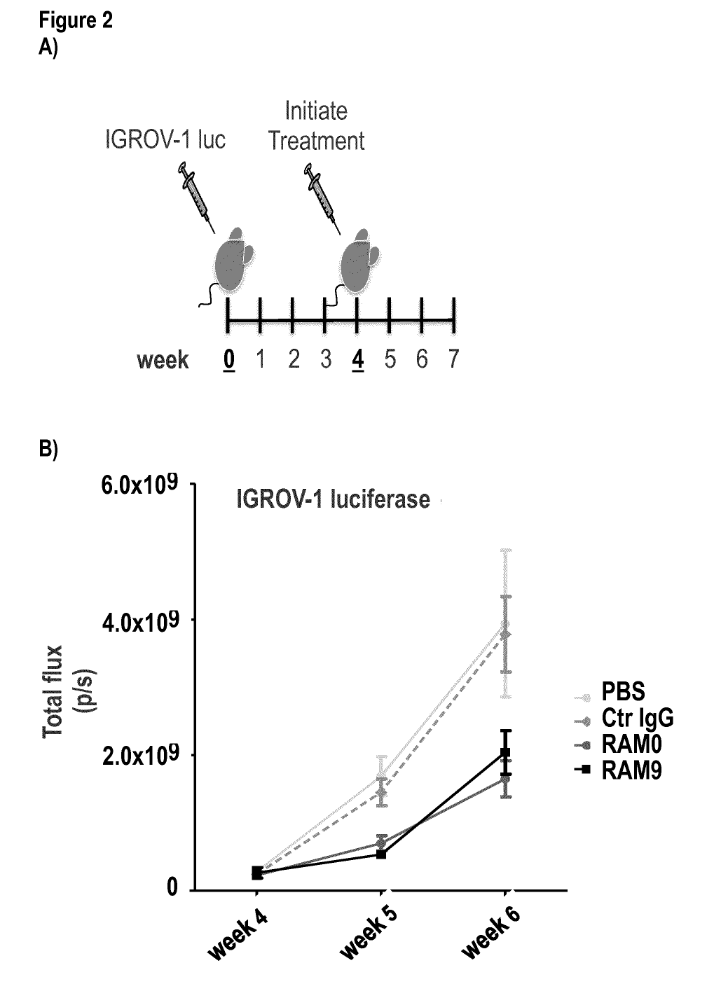

The results are shown in FIG. 2. As can be seen from the Figure, the treatment with both anti-MIF antibodies resulted in a reduction in luciferase activity. Thus, it was concluded that anti-MIF antibodies including RAM9 and RAM0 result in a reduction in tumor cell proliferation of TP53 mutant cancers.

### Example 3

### Treatment Effects of RAM9 and RAM0 in KRAS-mutant PC3 Tumor Xenografts in vivo

Next the inventors set out to further confirm the effects of anti-oxMIF antibodies in TP53 and RAS mutant cells, which were observed in vitro in Example 1, in an in vivo model.

Human prostate adenocarcinoma PC3 cells (containing a mutant KRAS gene encoding a K-ras G12V mutation, and containing a deletion mutation in the TP53 gene) were harvested from exponentially growing cultures, mixed with growth factor-depleted matrigel and inoculated subcutaneously into the right flank of Mf1 nude mice (2×10⁶ cells in 0.25 mL Matrigel/mouse, n=10/group). On the next day the antibody treatment was started (5 or 15 mg/kg intraperitoneally every other day). On day 7 after cell injection, the size of tumor xenograft was started to be measured and volume was calculated using the formula V=0.5 x a x b² (where “a” is the longest diameter; “b” is the shortest diameter. After termination of the experiment, tumors were excised, flash frozen and homogenized in lysis buffer. Cleared lysates were analyzed by multiplex immunoassays.

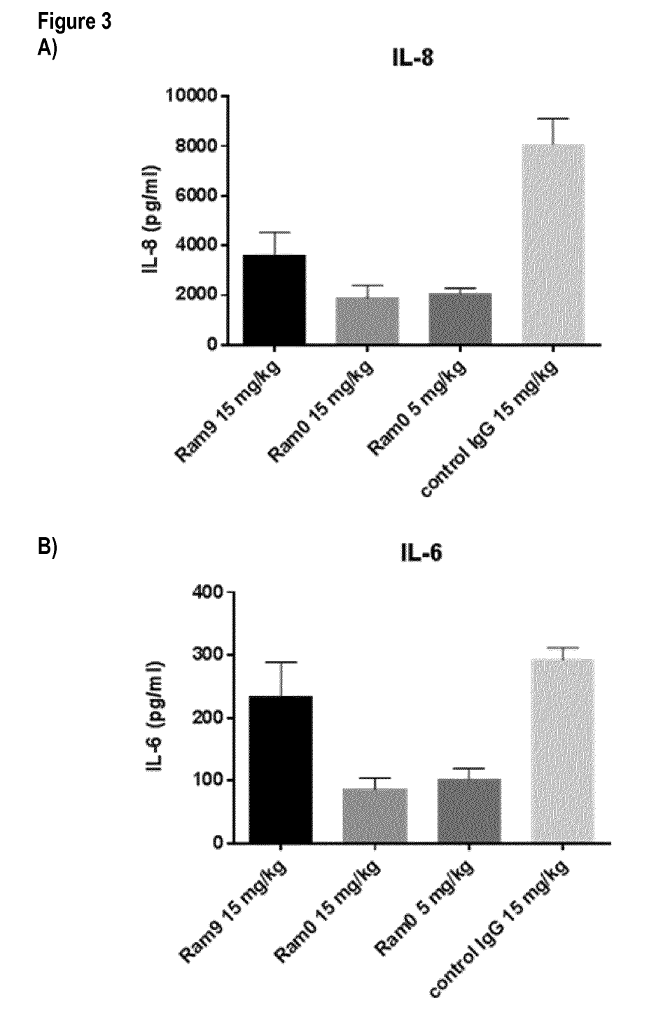

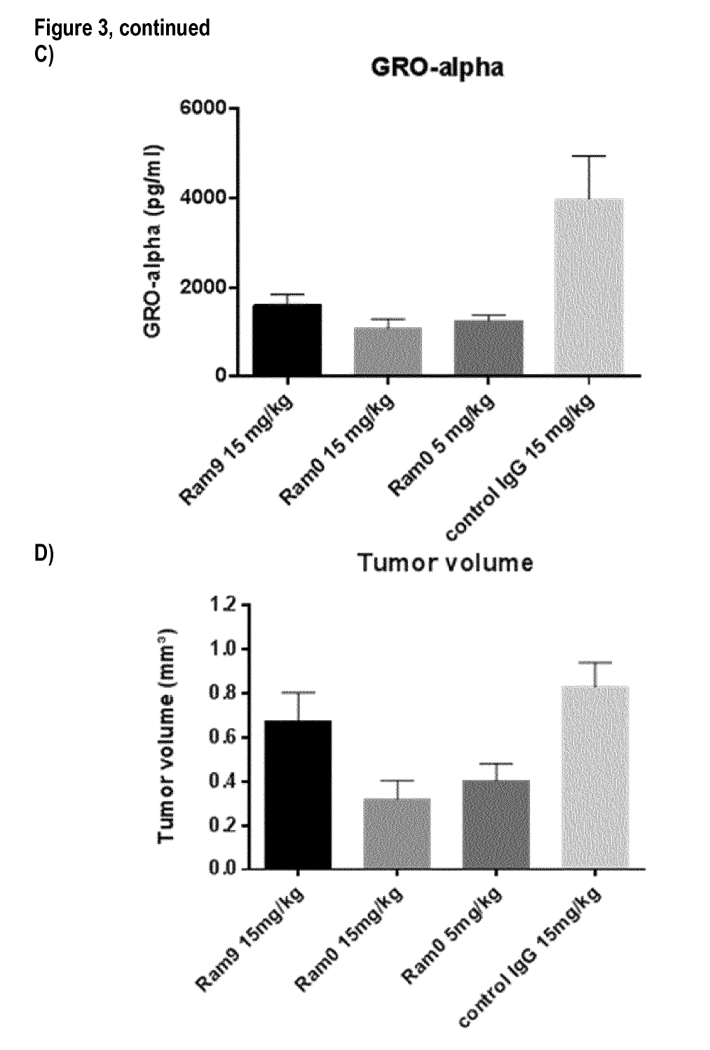

The results are shown in FIG. 3. Tumor volume (FIG. 3D) and intratumoral levels of the cytokines, 11-6 (FIG. 3B), II-8 (FIG. 3A) and GRO-alpha (FIG. 3C) are shown for the tumors from the PC3 prostate cancer xenograft model after treatment with the indicated antibodies. As can be seen from FIG. 3D, for both of the anti-MIF antibodies (RAM9 and RAM0), the tumor volume was decreased as compared to the control antibody. Additionally, the levels of the cytokines II-6, II-8 and GRO-alpha were reduced in the RAM9- and RAM0-treated tumors of the PC3 prostate cancer xenograft model (FIGS. 3A to 3C). The reduction of these cytokines indicates beneficial immunomodulatory effects of RAM9 and RAM0 in the PC3 tumor xenografts.

Thus, anti-MIF antibodies can advantageously be used for the treatment of RAS and/or TP53 mutant tumors.

1. An anti-MIF antibody for use in the treatment of cancer in a human patient, wherein the cancer contains mutant TP53 and/or mutant RAS.

2. The anti-MIF antibody according to claim 1 for the use according to claim 1, wherein the cancer contains mutant TP53 but not mutant RAS.

3. The anti-MIF antibody according to claim 1 for the use according to claim 1, wherein the cancer contains mutant RAS but not mutant TP53.

4. The anti-MIF antibody according to any of the preceding claims for the use according to any of the preceding claims, wherein the anti-MIF antibody is to be used in combination with a chemotherapeutic agent, which is preferably gemcitabine, mitoxantrone, cisplatin, capecitabine, 5-fluorouracil, leucovorin and/or doxorubicin.

5. The anti-MIF antibody according to claim 1 or 4 for the use according to claim 1 or 4, wherein the cancer contains mutant TP53 and mutant RAS.

6. The anti-MIF antibody according to any of the preceding claims for the use according to any of the preceding claims, wherein the cancer is selected from the following group: pancreatic cancer, ovarian cancer, prostate cancer, breast cancer, colorectal cancer, lung cancer and colon cancer, more preferred pancreatic cancer, colorectal cancer, prostate cancer and ovarian cancer.

7. The anti-MIF antibody according to claim 4 for the use according to claim 4, wherein the cancer is pancreatic cancer, preferably pancreatic carcinoma.

8. The anti-MIF antibody according to any of the preceding claims for the use according to any of the preceding claims, wherein the use is a use for treating effects caused by mutant RAS.

9. The anti-MIF antibody according to claim 8 for the use according to claim 8, wherein the effect caused by mutant RAS is cancer-induced inflammatory environment.

10. The anti-MIF antibody according to claim 8 for the use according to claim 8, wherein the effect caused by mutant RAS is angiogenesis.

11. The anti-MIF antibody according to claim 8 for the use according to claim 8, wherein the effect caused by mutant RAS is cancer cell proliferation.

12. The anti-MIF antibody according to claim 11 for the use according to claim 11, wherein the cancer cell proliferation is reduced through an induction of cancer cell apoptosis.

13. The anti-MIF antibody according to claim 8 for the use according to claim 8, wherein the effect caused by mutant RAS is cancer metastasis.

14. The anti-MIF antibody according to any of the preceding claims for the use according to any of the preceding claims, wherein the anti-MIF antibody is selected from the following group: anti-MIF antibody RAM9, RAM0 and/or RAM4.

15. The anti-MIF antibody according to any of claims 4 to 14 for the use according to any of claims 4 to 14, wherein the chemotherapeutic agent is gemcitabine.

16. The anti-MIF antibody according to any of claims 4 to 6 and 8 to 14 for the use according to any of claims 4 to 6 and 8 to 14, wherein the cancer is metastatic colorectal cancer containing mutant RAS, wherein the chemotherapeutic agent is 5-fluorouracil and leucovorin, wherein the anti-MIF antibody is preferably RAM9, and wherein the treatment is a third-line therapy.

17. The anti-MIF antibody anti-MIF antibody according to any of claims 4 to 6 and 8 to 14 for the use according to any of claims 4 to 6 and 8 to 14, wherein the anti-MIF antibody is RAM9, the chemotherapeutic agent is doxorubicin, optionally in combination with cisplatin, and the cancer is ovarian cancer.

18. The anti-MIF antibody anti-MIF antibody according to any of claims 4 to 6 and 8 to 14 for the use according to any of claims 4 to 6 and 8 to 14, wherein the anti-MIF antibody is RAM9, the chemotherapeutic agent is gemcitabine and the cancer is pancreas carcinoma.

19. The anti-MIF antibody according to any of claims 4 to 6 and 8 to 14 for the use according to any of claims 4 to 6 and 8 to 14, wherein the anti-MIF antibody is RAM0, the chemotherapeutic agent is doxorubicin, optionally in combination with cisplatin and the cancer is ovarian cancer.

20. The anti-MIF antibody according to any of claims 4 to 6 and 8 to 14 for the use according to any of claims 4 to 6 and 8 to 14, wherein the cancer is non-small cell lung cancer, and wherein the chemotherapeutic agent is docetaxel.

21. The anti-MIF antibody according to any of claims 4 to 6 and 8 to 14 for the use according to any of claims 4 to 6 and 8 to 14, wherein the cancer is metastatic colorectal cancer, wherein the chemotherapeutic agent is leucovorin, oxaliplatin and 5-fluorouracil, and wherein the treatment is a first-line therapy.

22. The anti-MIF antibody according to any of claims 4 to 6 and 8 to 14 for the use according to any of claims 4 to 6 and 8 to 14, wherein the cancer is metastatic colorectal cancer containing mutant RAS, and wherein the chemotherapeutic agent is capecitabine.

23. The anti-MIF antibody according to any of claims 4 to 6 and 8 to 14 for the use according to any of claims 4 to 6 and 8 to 14, wherein the anti-MIF antibody is RAM0, the chemotherapeutic agent is gemcitabine and the cancer is pancreas carcinoma.

24. The anti-MIF antibody according to any of claims 4 to 6 and 8 to 14 for the use according to any of claims 4 to 6 and 8 to 14, wherein the chemotherapeutic agent is doxorubicin, optionally in combination with cisplatin and the cancer is ovarian cancer.

25. The anti-MIF antibody according to any of claims 4 to 6 and 8 to 14 for the use according to any of claims 4 to 6 and 8 to 14, wherein the anti-MIF antibody is RAM4, the chemotherapeutic agent is gemcitabine, and the cancer is pancreas carcinoma.

26. The anti-MIF antibody according to any of claims 4 to 6 and 8 to 14 for the use according to any of claims 4 to 6 and 8 to 14, wherein the anti-MIF antibody is RAM0, the chemotherapeutic agent is mitoxantrone, and the cancer is prostate cancer.

27. The anti-MIF antibody according to any of claim 1 or 3-26 for the use of any of claim 1 or 3-26, wherein the cancer contains mutant KRAS.


SEQUENCE LISTING (14 sequences)

SEQ ID NO: 1 - protein, 214 aa, Artificial Sequence
  Light chain of RAB9
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 214 aa, Artificial Sequence
  Light chain of RAB4
DIQMTQSPGTLSLSPGERATLSCRASQGVSSSSLAWYQQKPGQAPRLLIYGTSSRATGIPDRFSGSASGTDFTLTISRLQPEDFAVYYCQQYGRSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 214 aa, Artificial Sequence; identical to SEQ ID NO: 2
  Light chain of RAB0

SEQ ID NO: 4 - protein, 214 aa, Artificial Sequence
  Light chain of RAB2
DIQMTQSPVTLSLSPGERATLSCRASQSVRSSYLAWYQQKPGQTPRLLIYGASNRATGIPDRFSGSGSGTDFTLTISRLEPEDFAVYYCQQYGNSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 445 aa, Artificial Sequence
  Heavy chain of RAB9
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 454 aa, Artificial Sequence
  Heavy chain of RAB4
EVQLLESGGGLVQPGGSLRLSCAASGFTFSIYAMDWVRQAPGKGLEWVSGIVPSGGFTKYADSVKGRFTISRDNSKNTLYLQMNSLRAEDTAVYYCARVNVIAVAGTGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 454 aa, Artificial Sequence
  Heavy chain of RAB0
EVQLLESGGGLVQPGGSLRLSCAASGFTFSWYAMDWVRQAPGKGLEWVSGIYPSGGRTKYADSVKGRFTISRDNSKNTLYLQMNSLRAEDTAVYYCARVNVIAVAGTGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 454 aa, Artificial Sequence; identical to SEQ ID NO: 6
  Heavy chain of RAB2

SEQ ID NO: 9 - protein, 457 aa, Artificial Sequence
  RAM0hc
EVQLLESGGGLVQPGGSLRLSCAASGFTFSWYAMDWVRQAPGKGLEWVSGIYPSGGRTKYADSVKGRFTISRDNSKNTLYLQMNSLRAEDTAVYYCARVNVIAVAGTGYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence; identical to SEQ ID NO: 2
  RAM0lc

SEQ ID NO: 11 - protein, 448 aa, Artificial Sequence
  RAM9hc
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence; identical to SEQ ID NO: 1
  RAM9lc

SEQ ID NO: 13 - protein, 457 aa, Artificial Sequence
  RAM4hc
EVQLLESGGGLVQPGGSLRLSCAASGFTFSIYAMDWVRQAPGKGLEWVSGIVPSGGFTKYADSVKGRFTISRDNSKNTLYLQMNSLRAEDTAVYYCARVNVIAVAGTGYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Artificial Sequence; identical to SEQ ID NO: 2
  RAM4lc